(19) United States
(12) Patent Application Publication
(10) Pub. No.: US 2007/0184546 A1
(43) Pub. Date: Aug. 9, 2007

(54) Systems and methods for automated proteomics research

(75) Inventors: Philip J. Farrelly, Short Hills, NJ (US); Scott Bartolett, Bridgewater, NJ (US); Steven Riedmuller, Roseland, NJ (US)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Hudson Control Group, Inc., Springfield, NJ (US)

(21) Appl. No.: 11/605,611

(22) Filed: Nov. 29, 2006

Related U.S. Application Data
(60) Provisional application No. 60/742,109, filed on Dec. 2, 2005.

(51) Int. Cl. C12M 1/34

(52) U.S. Cl. 435/286.3; 435/303.1; 435/309.1; 435/287.3

(57) ABSTRACT

A robotic laboratory automation workcell preferably includes instruments and equipment that are integrated by using conveyor or track elements and a robotic arm. The automation workcell is controlled by a centralized or main controller or processor using specialized control software to automate the proteomics research process. The automated workcell is capable of performing genetic laboratory experiments from start to finish by moving samples or microplates between the instruments for analysis. A goal of the automated workcell is to perform repetitive procedures in an effort to build and maximize the efficiency of a gene(s) of a targeted organism.

### CROSS REFERENCE TO RELATED APPLICATIONS

This application claims the benefit of the filing date of United States Provisional Patent Application No. 60/742,109 filed Dec. 2, 2005, the disclosure of which is hereby incorporated herein by reference.

The U.S. government has a paid-up license in this invention and the right in limited circumstances to require the patent owner to license others on reasonable terms as provided for by the terms of Contract No. 58-36204-146 awarded by the U.S. Department of Agriculture, Agricultural Research Service. Contract No. 58-36204-146 is a joint research agreement between Hudson Control Group Inc. and U.S. Department of Agriculture, Agricultural Research Service executed on Jul. 6, 2004 relating to the following field of the invention.

### FIELD OF THE INVENTION

The present invention relates to genetic research and drug discovery research. The invention involves systems and methods for automating the growing and processing of cells to reproduce genetic materials and their resultant proteins and for measuring or analyzing the results.

### BACKGROUND OF THE INVENTION

Pharmaceutical, biotechnology and agribusiness companies have a constant need to grow cells of various organisms, such as Escherichia coli bacteria, yeasts, plant, mammalian cells, etc., in order to assess the effects of chemical compounds and/or genetic materials on the health of these organisms or upon the production of organic products, such as proteins, or to improve or optimize cell lines of useful organisms, such as yeasts. This type of research requires the processing of tens, or even hundreds, of thousands of individual cell colonies, often in a repetitive manner. The ultimate goal is to detect minor variations in the resulting effects of the compounds or genetic materials, then to alter the compounds or genetic materials in an attempt to optimize the sought-after results.

Due to the large number of samples to be tested, these processes usually utilize 96-well or 384-well microplates, each well containing a different sample to test, or a unique set of samples, and instruments that automate the processing of these microplates. But, even concentrating the test format into the footprint of a microplate still requires the processing of potentially thousands of microplates before success can be achieved.

For example, in a process of genetic research the researcher tries to assess the function of a gene and/or modify a gene to improve its function. This process may have many steps that need to be performed and includes different types of laboratory equipment. It is very labor intensive because the researcher needs to move materials between work stations and machines manually. Manual movement is disfavored due to an increase in the rate of contamination. Moreover, manual movement between experiments and workstations may increase the rate of human error due to spillage and dropping invaluable experimental samples or simple confusion or processing errors.

Currently, typical automation equipment used in laboratory processes include automated colony-picking robots, robotic pipettors, automated plate seal applicators, and automated liquid dispensers. However, automation practitioners have been heretofore unable to produce a comprehensive system that will perform labor intensive and repetitive procedures associated with all or multiple steps in the laboratory process. A lack of the required software tools, inadequate microplate delivery systems and insufficient knowledge of practicable methods of integrating necessary components have prevented such substantial integrations from being implemented previously.

### SUMMARY OF THE INVENTION

The invention relates to an automated system with centralized control for performing proteomics research in one or more workcells.

In one embodiment, the system is a workcell that is configured to perform automated transfer of microplates between a colony picking robotic device and a liquid handling and/or pipetting robot for proteomics research including, for example, plasmid preparation.

In another embodiment, the system is a workcell that is configured for the automated transfer of microplates between a colony picking robotic device and an incubator suitable for prokaryotic and eukaryotic cell cultivation in microplates.

In another embodiment, the system is such a workcell that is further configured to include the automated transfer of microplates to and from an automated microplate seal applicator.

In another embodiment, the system is such a workcell that is further configured for the automated transfer of microplates to and from a liquid handling and/or pipetting robot.

In another embodiment, the system is a workcell that is configured for automated transfer of microplates between a colony picking robotic device and a workcell or workstation configured for automated preparation of plasmids for use in genomic or proteomic processes.

In another embodiment, the system is such a workcell that is further configured for the automated transfer of microplates to and from a workcell or workstation that performs automated preparation of plasmids for use in genomic or proteomic processes.

In another embodiment, the system as described in any of the preceding embodiments is configured with a scheduling control device or software and control processor configured to control and operate the equipment of the workcells.

In still another embodiment, the scheduling device or software of the previously described embodiments is configured to control and operate the equipment of the workcell in a manner that permits multiple workcells or equipment of each workcell to be operating simultaneously, processing the same or different sets of samples.

In such automated embodiments, systems may be configured to automate processing of bacterial, yeast or other microbial colonies from their initial plated colony growth through their picking into microplate wells, subsequent growth, archiving, plasmid preparation, plasmid quality analysis, plasmid-based reactions and assays without manual intervention. Futhermore, the above embodiments may be configured to automate the identification of novel open reading frames (DNA segments) through a strategy of mutagenizing wild-type genes in order to introduce or improve protein production characteristics in lines of bacteria, yeast and/or all eukaryotic cells, including but not limited to plant callus cultures, and mammalian, reptilian, amphibian, arthropodian, and protozoan cells. Moreover, such systems may be configured for assembling open reading frames sequentially to form full-length genes via polymerized chain reaction (PCR) process to include any known or desired codon sequence pattern in particular open reading frames. The embodiments may be further configured with centralized control to implement assembling open reading frames to form full-length genes via PCR process to include both wild-type and optimized or improved open reading frames identified using the described workcells. Such automated control may include workcells for transforming or transfecting the assembled gene structures produced into prokaryotic or eukaryotic cells, including bacterial, yeast, plant or animal cells.

In one embodiment, an automated system implements a method for modifying an ORF of a gene so that the expression product of said ORF is characterized by a desirable functional modification in the automated steps of incrementally synthesizing a plurality of progressively larger segments of said ORF wherein at least one of said segments comprises an introduced modification; simultaneously expressing each of said segments in an expression system; determining the biochemical activity and/or binding site recognition of each of said segments; and selecting at least one of said segments characterized by said desirable functional modification based on said biochemical activity and/or binding site recognition. Optionally, a plurality of progressively larger segments of said ORF may be in at least one to infinite numbers of combinations. Optionally, the expression may be either from cDNA libraries or from modified ORFs. In such an automated system, the production of new bacterial and/or fungal strains may be performed by mass transformation and/or transfection of eukaryotic cell lines with said cDNA libraries.

In another such embodiment, the automated system is configured for automatically modifying an ORF of a gene so that the expression product of said ORF is characterized by a desirable functional modification or combination of functional modifications in automated steps of (a) providing a plurality of clones having a first introduced modification in an ORF as compared to a wild-type form of said ORF; (b) mutagenizing the plurality of clones in order to introduce at least one additional modification into the ORF of each one of said plurality of clones; (c) simultaneously expressing the ORF in each one of the clones from step (b) in an expression system; (d) determining the biochemical activity and/or binding site recognition of each of the clones; and (e) selecting a clone having the desirable functional modification or combination of functional modifications based on the biochemical activity and/or binding site recognition of the clone.

The system may perform specific methods of automated proteomics research.

One embodiment includes a method for selecting desirable functional modification of an ORF. This means that larger segments of an ORF are progressively and incrementally synthesized. The ORF may be naturally occurring (wild-type) sequence or have at least one introduced modification. First, each segment is expressed in an expression system. Then, the biochemical activity and/or binding site recognition of each ORF are determined. Last, desirable functional modification based on the biochemical activity and/or binding site recognition of one of a segment having an introduced modification is selected. This method may be performed by incrementally synthesizing by means of amplifying overlapping oligomers where the oligomers collectively represent the sequence of the entire ORF. The method of the present invention may be performed in vitro, in vivo, in vivo in a bacterium, in vivo in yeast, and both in vitro and in vivo.

In another embodiment the method is for producing a clone having one or more desirable functional modifications of an ORF. Steps in performing this method include incrementally synthesizing progressively larger segments of an ORF in any direction on the ORF, either on the wild-type sequence or at least one of the segments with a first introduced modification. Then, express the segments in an expression system. Next, determine the biochemical activity and/or binding site recognition of each of the segments. Further, select for the desirable functional modification based on the biochemical activity and/or binding site recognition of the segment having a first introduced modification in the ORF. Then, incrementally synthesizing progressively larger segments of the ORF having the first introduced modification where at least one of the segments has a second introduced modification. Finally, express the segments in an expression system, and determine the biochemical activity and/or binding site recognition of each of said segments, selecting for the desirable functional modification based on the biochemical activity and/or binding site recognition of the segment with both the first and second introduced modifications. The segments of ORFs may be a wild-type ORF of a gene. The segment may encode a reduced number of translational stops of any type when expressed in a heterologous expression system as compared to a wild-type ORF of said gene. In this embodiment the desirable biochemical activity and/or binding site recognition may be screened for high solubility and greater ability to be purified from the aqueous fraction. The desirable biochemical activity and/or binding site recognition are screened for reduction in formation of cysteine bridges by systematic removal of all cys amino acids in the ORF. The segment or ORF may be mixtures of wild-type and modified segments of sequences in the assembled ORF and the ORF may be optimized to incorporate codons most frequently used in a particular organism expression system.

In an embodiment, a method is for modifying an ORF of a gene so that the expression product of said ORF is characterized by a desirable functional modification using the following steps. First, incrementally synthesizing progressively larger segments of the wild-type ORF or an ORF having at least one introduced modification. Next, each of the segments are expressed in an expression system and their biochemical activity and/or binding site recognition of each segment is determined. Finally, a selection for the desirable functional modifications based on the biochemical activity and/or binding site recognition of the segment having an introduced modification may be determined. This embodiment may include a truncated form of a wild-type ORF of the gene. Further, in this embodiment the segment may encode a reduced number of translational stops of any type when expressed in a heterologous expression system as compared to a wild-type ORF of the gene. Additionally in this embodiment the desirable biochemical activity and/or binding site recognition is screened for high solubility and greater ability to be purified from the aqueous fraction. In this embodiment the desirable biochemical activity and/or binding site recognition is screened for reduction in formation of cysteine bridges by systematic removal of all cys amino acids in the ORF. In this embodiment the segment could be mixtures of wild-type and modified segments of sequence in the assembled ORF.

In any of the above embodiments the method may be used to optimize the entire ORF to incorporate the codons that are most frequently used in a particular organism expression system.

In another embodiment, the method may be for producing a clone having a plurality of desirable functional modifications of an ORF including the steps of first providing a clone having a first introduced modification in an ORF as compared to a wild-type form of the ORF. Then, introducing at least one additional modification into the ORF. Further, expressing the ORF in an expression system, determining the biochemical activity and/or binding site recognition of the ORF and selecting for the desirable functional modifications based on the biochemical activity and/or binding site recognition of the ORF. In this embodiment there may be an introduction of at least one additional modification selected by random mutagenesis, directed mutagenesis, and evolutionary mutagenesis. The desirable clone may be subjected to a mutagenesis process for a second time.

In an alternative embodiment, the invention may be used in a method for obtaining a representative ORF for each gene (all or partial) in the genome of an organism (bacterial or fungal). The genes may be derived from collections of cDNAs in various previously generated libraries. First, an ORF is provided for each gene in the cDNA library capable of being expressed as a protein in the system. Then, The ORF is expressed in an expression system. Further, the cDNA libraries are screened for the expressed proteins for desired function in vivo or in vitro by determining binding and/or biochemical activity. Last, the biochemical activity and/or binding site recognition of the ORFs from the cDNA library is determined.

In another embodiment, the method may be used to introduce a modification in at least one ORF from a cDNA library selected by random mutagenesis, directed mutagenesis, and evolutionary mutagenesis. Then, subsequently selecting for desirable functional modifications based on the biochemical activity and/or binding site recognition of the expressed ORFs.

An additional uses of the present invention may involve a method for transforming and/or transfecting an entire library of cDNAs having a representative ORF for each gene (all or partial) in the genome being expressed in prokaryotic and/or eukaryotic cells. First, an ORF in a cDNA library is provided for each gene capable of being expressed in the prokaryotic, and/or eukaryotic cells. Then, the ORF from the cDNA library is expressed in an expression system. The cDNA libraries are screened for their expressed protein and desired function in vivo or in vitro by determination of binding site recognition and/or biochemical activity of the cDNA library ORF.

Another use of the invention may be to transform an entire cDNA library of modified ORFs from assembly. This is done by having at least one representative ORF for a mutagenized gene being expressed. First, an ORF for each gene capable of being expressed in the system is provided. Then, the ORF is expressed in an expression system. Further, the cDNA cDNA libraries are screened for the expressed protein and for desired function in vivo or in vitro by determining binding and/or biochemical activity. Last, the biochemical activity and/or binding site recognition of the cDNA library ORF is determined and a selection for the desirable functional modifications based on the biochemical activity and/or binding site recognition of the ORF is performed. The cDNA libraries for high throughput transformation of prokaryotic and fungal strains and/or eukaryotic cell lines are of either wild-type, random mutagenized, targeted mutations, and. or evolutionary mutations are present in combinations or separately.

In an alternative embodiment, it may be used in a method for using high throughput screening to assess said ORFs and the transformed strains and/or transfected cell lines. First, the transformed strain and/or transfected cell line is obtained. Second, the strain is screened in high throughput using an appropriate assay such as a growth assay, binding assay and/or biochemical assays to identify ORFs and strains with optimal characteristics.

Additional aspects of the invention will be apparent from a review of the following disclosure and claims.

### BRIEF DESCRIPTION OF THE DRAWINGS

FIG. 1 is a representation of an example configuration of an automated proteomics workcell;

FIG. 1a is an example of a robotic arm that may be integrated for use in the workcell;

FIG. 1b illustrates various track elements for integrating laboratory automation equipment;

FIG. 1c is an illustration of a connector that connects two track elements together;

FIG. 1d is a representation of a plate stacker element;

FIG. 1e is an illustration of a plate stacker element expander and connection devices;

FIG. 1f is an illustration of a device for connecting and integrating an incubator into the workcell;

FIG. 1g is an illustration of a plate sensor and a plate holding device;

FIG. 1h is an illustration of an automated workcell configured with two track elements on angle wherein a robotic arm can deliver plates between the two track elements;

FIG. 1i is an illustration of a workcell configuration;

FIG. 1j is an illustration of the workcell in FIG. 1i in a modified configuration;

FIG. 1k is an illustration of a small scale workcell;

FIG. 2 is a block diagram control schematic of a controller for controlling an automated proteomics workcell;

FIG. 3 is a block diagram representation of the control system for a component integrated into the automated proteomics workcell;

FIG. 4 is an example user interface for configuring elements of an automated proteomics workcell;

FIG. 5 is an example user interface for configuring a specific component of the automated proteomics workcell;

FIG. 6 is an example user interface for plate setup with the configuration software;

FIG. 7 is an example user interface of a method editor with the configuration software for an automated proteomics workcell;

FIG. 8 is an example user interface for executing a workcell process with the configuration software of the automated system;

FIG. 9 is a user interface showing process control setup of the configuration software; and

FIG. 10 is a user interface that illustrates a predictive scheduler of the configuration software.

FIG. 11 shows automated system including stacker units, a pick-and-place robot arm and two additional instruments integrated in the system including an ELX-405 Plate Washer from BioTek and a MultiDrop Dispenser from T hermoLabSystems.

### DETAILED DESCRIPTION

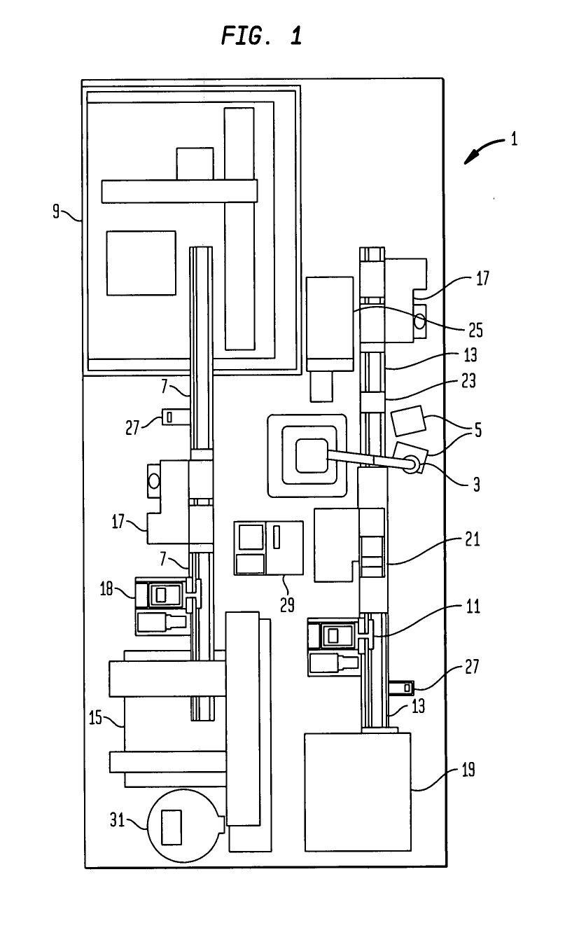

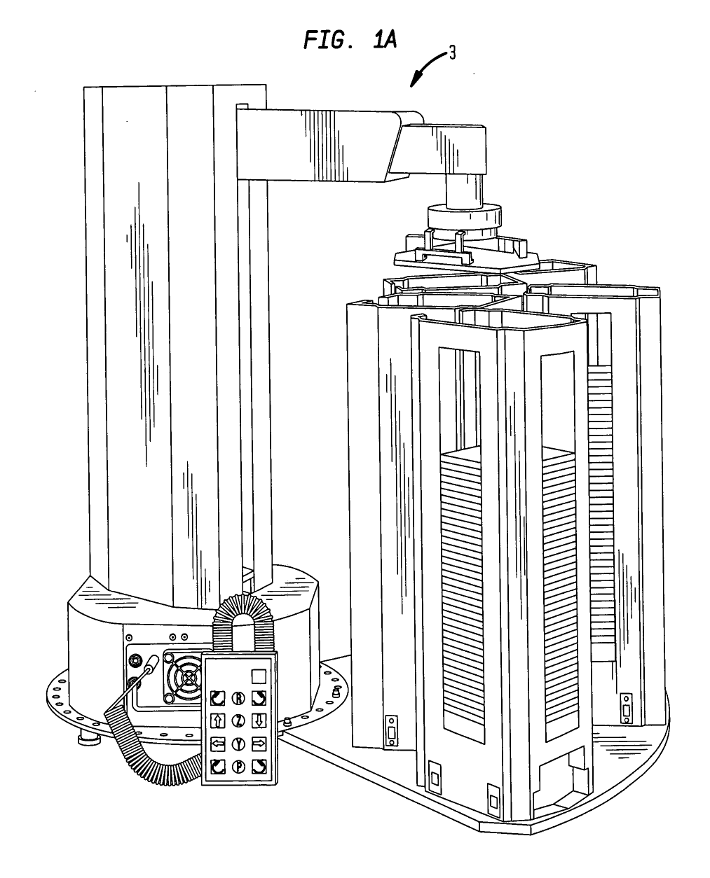

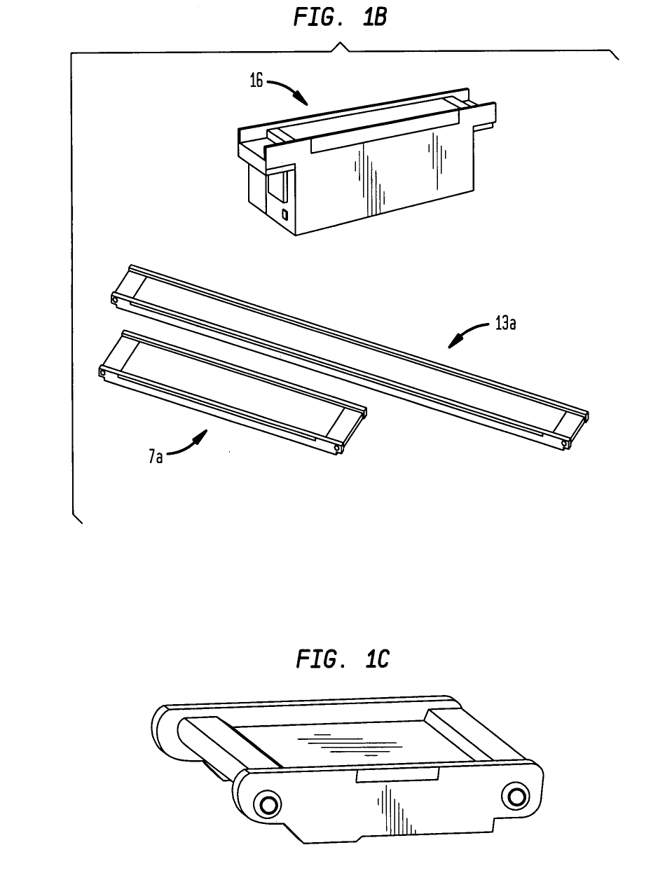

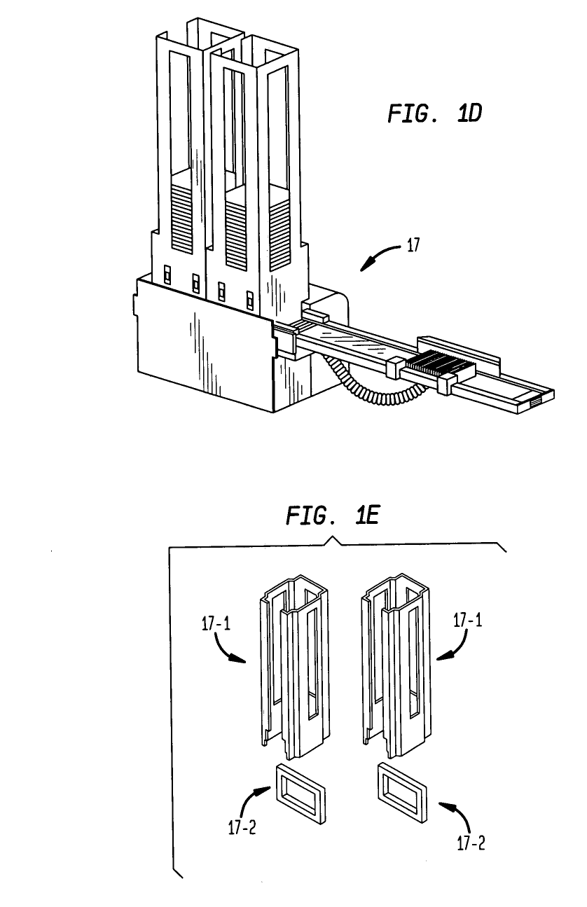

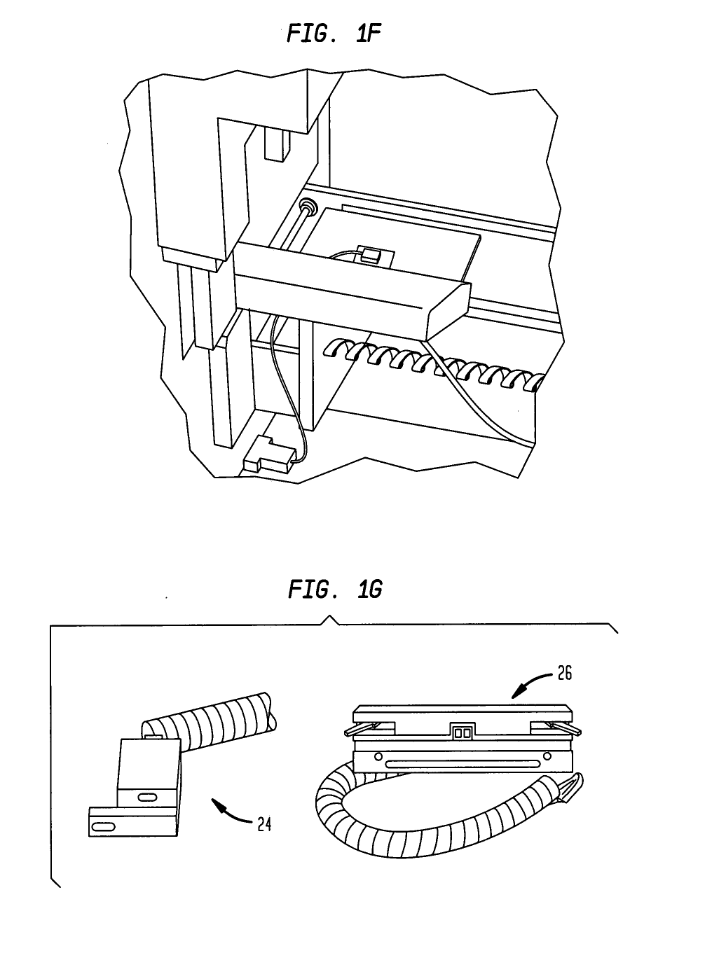

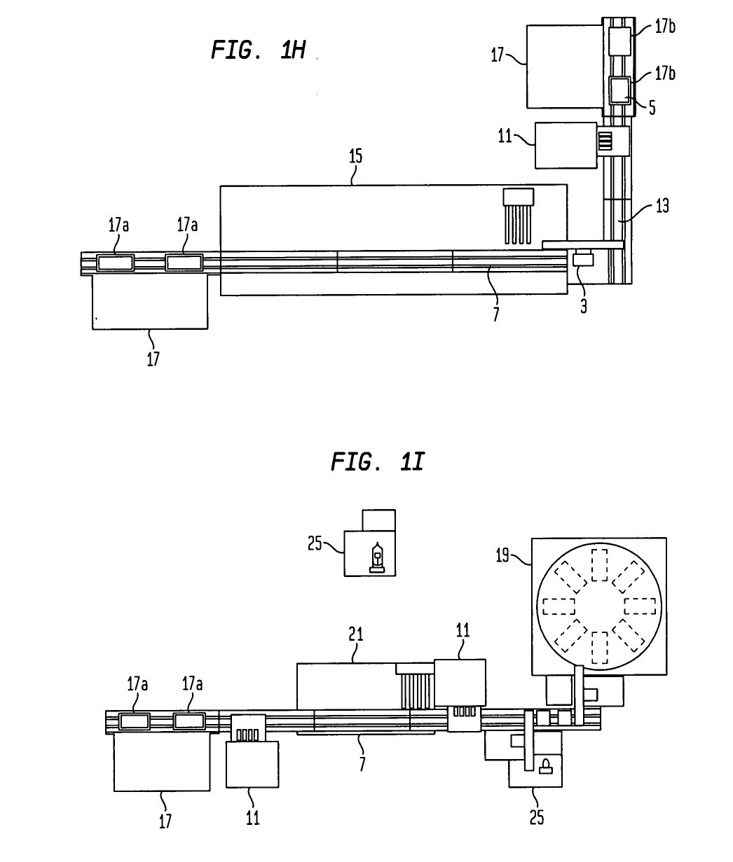

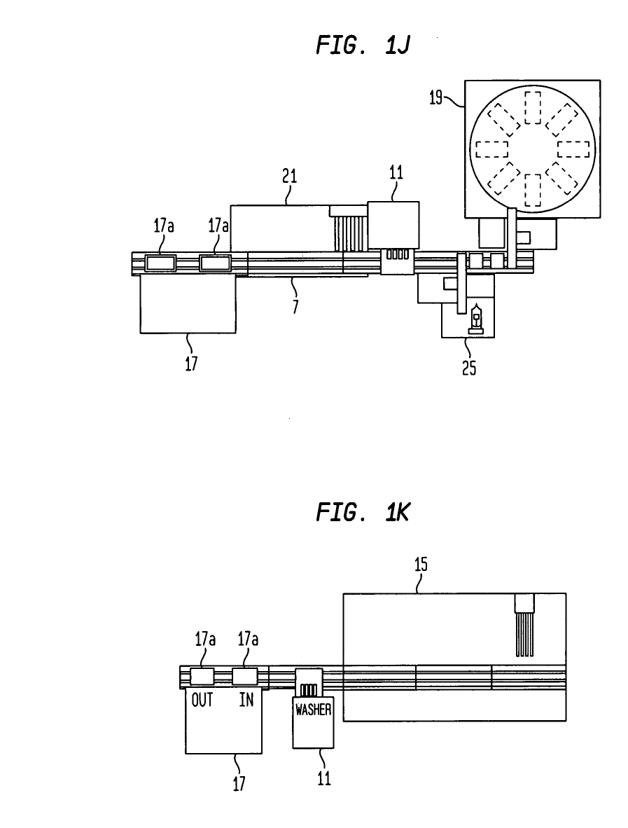

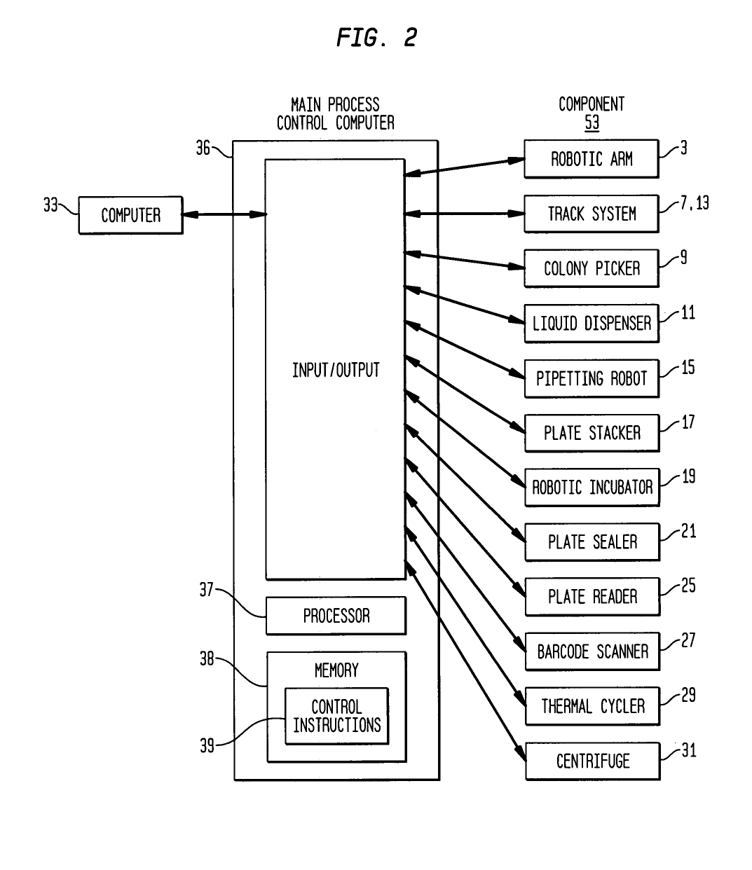

Referring to the figures, where like numerals indicate similar features, FIG. 1 illustrates an automated proteomics workcell 1 for a genetics laboratory. The workcell 1 preferably includes several instruments integrated and adapted for automated manipulation of a laboratory specimen. For example, as illustrated in FIG. 1 such integration may be achieved by implementation of robotic arm 3 and/or track elements 7, 13 or by any other means of automated microplate transport between devices that work with samples or microplates in an automated proteomics process. The devices of such a system are preferably controlled by a centralized controlling unit illustrated in FIG. 2.

As shown in FIG. 1 a preferred embodiment of the automated proteomics workcell 1 has a colony picker element 9, one or more reader elements 27 such as barcode reader, one or more plate stacker elements 17, a 96-well aspirator 18, a pipetting robot 15, a robot arm element 3, a plate sealer element 21, a liquid handler element 11, a robotic incubator element 19, a thermal cycler element 29, a centrifuge element 31, a plate reader element 25, and microplate track elements 7, 13. The laboratory instruments, such as the colony picker element 9, 96-well aspirator 18, pipetting robot 15, plate sealer element 21, liquid handler element 11, robotic incubator element 19, thermal cycler element 29, centrifuge element 31, and plate reader element 25, are integrated through track elements 7, 13 and/or a robotic arm 3, and the automation process is controlled by a controller such as the one illustrated in FIG. 2.

In a preferred embodiment the colony picker element 9, plate stacker element 17, 96-well aspirator element 18, pipetting robot element 15, and centrifuge element 31 lie along one track element 7. A plate stacker element 17, plate sealer element 21, liquid dispenser element 11, and robotic incubator element 19 lie along a second-track element 13. The plate reader element 25, robotic arm element 3, and thermal cycler element 29 lie in between the two track elements 7, 13. Therefore, in a preferred configuration two track elements 7, 13 are used. The robot arm element 3 controls movement of samples or microplates between the two track elements 7, 13.

Suitable instruments for the system are available equipment from third parties and are adaptable to allow for integration into the workcell 1. Equipment from third parties may contain firmware/software that allows an individual component to run a process with a sample separately. Firmware/software of the components may be modified so that an individual component will communicate with the central controller of the workcell 1 as discussed in more detail herein and as illustrated by FIG. 2.

For example, advanced liquid handling systems 11 that may be integrated into the robotic workcell include Beckman Coulter Biomek® 2000, Beckman Coulter Biomek® FX, Cartesian Ursys 2000, Colibri Plato 7, Hamilton Microlab© 4200, Perkin Elmer Lifesciences (Packard) MultiProbe® (all models), Perkin Elmer Lifesciences (Packard) PlateTrak/MiniTrak, PerkinElmer Lifesciences (Packard) Evolution™ P3, Qiagen (Rosys) Winrufas—Plate, Sias Xantus, Tecan Genesis, Xiril, Zymark RapidPlate®96. Pipetting robots 15 that may be integrated into the robotic workcell include Apricot TPS-384, Beckman Coulter Multimek™/Multipette, Bio-Tek® Precision 2000, CyBio Cybi™-Well, Gilson 215, Matrix PlateMatePlus™, Matrix PlateMate (old style), Matrix SerialMate™, PerkinElmer Life Sciences Apricot Pipettors, Robbins Hydra® 96, Robbins Hydra® 384, Robbins Tango™, Tecan Genmate, TomTec Quadra, Velocityll VPrep™. Washers that may be integrated to work with the colony picker 9 include Bio-Tek® EL404, Bio-Tek® ELX405, Digital Imagers Flusher, Molecular Devices Embla 384, Molecular Devices Embla 96/384, Tecan Columbus, Tecan PW 384. Dispensers that may be integrated into the robotic workcell 1 include Bio-Tek MicroFill AF1000, Genetix QFill2, Molecular Devices AQUAmax™ 96/384 & 1536, Thermo Labsystems Multidrop 384, Thermo Labsystems MultiDrop DW. Readers/imaging systems 25 that may be integrated into the robotic workcell 1 include Amersham Biosciences LEADseeker™, Amersham Biosciences LEADseeker Multimodality, Amersham Biosciences INCell Analyzer 1000 and 3000, Applied BioSystems CytoFluor® 4000, Aurora Biosciences VIPR™ II, BD Biosciences FACSCalibur Flow Cytometer with Cytek Autosampler, Bio-Rad UltraMark, Bio-Tek® ELX808, Bio-Tek® FL600, Bio-Tek® PowerWaveX, Bio-Tek® PowerWaveX 340, Bio-Tek® PowerWave HT, Bio-Tek® Synergy HT, BMG FLUOstar, BMG NEPHELOstar, BMG POLARstar, Imaging Research AutoLead™, Molecular Devices Analyst™ HT, Molecular Devices CLIPR™, Molecular Devices FlexStation™, Molecular Devices FLIPR, Molecular Devices SPECTRAmax® 190, Molecular Devices SPECTRAmax 250, Molecular Devices SPECTRAmax Plus, Molecular Devices SPECTRAmax Gemini, Molecular Devices Vmax, Molecular Devices ThermoMax, Molecular Devices VERSAmax™, Perkin Elmer Lifesciences (Packard) AlphaQuest™, Perkin Elmer Lifesciences (Packard) Fusion™, PerkinElmer Life Sciences Victor 1,2,2V, PerkinElmer Life Sciences Wallac Jet, PerkinElmer Life Sciences Wallac Trilux, PerkinElmer Life Sciences/Wallac ViewLux, PerkinElmer HTS 7000, Q3DM EIDAQ 100 High Throughput Microscopy Platform, Tecan SAFIRE, Tecan SPECTRAFluor, Tecan SPECTRAFluor Plus, Tecan SPECTRA Image, Tecan ULTRA, Thermo Labsystems Fluroskan Ascent FL, Thermo Labsystems Multiskan Ascent, Universal Imaging Corporation Discovery-1, Arryx laser microscopes, Zeiss HTS-Reader. Bar code labelers/scanners 27 that may be integrated into the robotic workcell 1 include Beckman Coulter Sagian™ Print & Apply, Computype LAP-4100 600 DPI Printer-Applicator, Keyence BLO600 Scanner, Microscan MS-710 Scanner, Symbol LS 1220 Scanner, Symbol LS 6804 Scanner, Velocity 11 VCode™, Zymark Presto™ Labeling Workstation. Sealers 21 that may be integrated into the robotic workcell 1 include ABgene ALPS 300 Plate Sealer, ABgene ALPS Plate Sealer, Remp Plate Sealer, Velocity 11 PlateLoc™ Thermal Plate Sealer. A thermal cycler 29 that may be integrated into the robotic workcell 1 includes MJResearch PTC series. Mass Spec machines 25 that may be integrated into the robotic workcell include MicroMass®, and Thermo Finnegan Xcalibur™. Incubators/Freezers/Storage units 19 that may be integrated into the robotic workcell 1 include Jouan Robotics MolBank™, Kendro® Heraeus® Cytomat, Kendro® Heraeus® Cytomat 2, Liconic STX Series/Jouan Robotics AutoCell and NBS (New Brunswick Scientific) Innova 4230. Microarrayers that may be integrated into the robotic workcell 1 include Brown Microarrayer and Radius 3XVP Arrayer.

Overall, it is optimal to have microplate compatible based instruments for integration into the workcell 1. Therefore, equipment that may be modified to fit a track element 7, 13 is preferred. For example, it is optimal when a washer component is modified such that the track element may fit into the work area of the washer, allowing the microplate to stay on the track elements while the component operates. This eliminates the need to use an external instrument to move the plate, increasing throughput, simplicity, and reliability. Other instruments of the system are preferably modified in this way.

In a preferred embodiment, the system is used for performing general gene studies. For example, this may include plasmid preparation, restriction mapping, in vitro protein expression, sample assay, and mass transformation of prokaryotic or fungal or yeast cells and/or transfection of eukaryotic cells. However, the invention is not limited to basic genetic laboratory experimentation procedures mentioned herein. The invention may be catered to specific laboratory needs.

In the following discussion, a basic gene study will be used by example only to demonstrate how the components of the workcell 1 function together with common or integrated automated control. The invention may be used to perform gene studies by first preparing growth dishes that may be introduced to the fully automated proteomics workcell 1. This may be done by taking genes from a cDNA library and the mutants and analogues thereof, and inserting these fragments into plasmids. The plasmids are then placed into E. coli cells using known laboratory techniques. The E. coli cells are then grown on proper medium within the growth dishes. Growth typically starts on growth dishes wherein the individual colonies, each containing a unique gene insert in their cells' plasmids, develop spatially separated on the surface of the solid medium in the growth dish. Growth dishes are then introduced into an automated proteomics workcell system 1 in magazines of growth dishes 5 accessed by a benchtop robot arm 3. Alternatively, the magazines or growth dishes may be introduced to the system via a plate stacker 17, which will deliver the growth dishes into the workcell 1 via a track system 7. Multiple plate stackers 17 may be used to increase capacity in the system and in the preferred embodiment two plate stackers 17 are used.

The robotic arm 3 illustrated in FIG. 1a and is preferably configured for automated control in a plurality of ranges of motion. For example, the arm extends or telescopes in all directions along the X and Y axes. In addition, the robotic arm 3 is capable of movement around its central point to allow for rotation of the arm mechanism. The robotic arm has its own control unit and can be set up and trained to recognize pickup and drop-off coordinates of the growth dishes or magazines. A training system for the robotic arm 3 is described in U.S. patent application Ser. No. 10/945,196 which is incorporated herein by reference. The robotic arm 3 may move the magazines or growth dishes 5 on, off and between track elements 7, 13.

The track elements 7, 13 can be described as a conveyor and are illustrated in FIG. 1b. The track elements 7, 13 are used to configure the workcell 1 and integrate machinery so that all experimentation processes of components of the system may be automated to work on the same microplates moving among the instruments of the system. Track elements 7, 13 may be the tracks from the LabLinks system made by Hudson Control Group. Track elements 7 and 13 are movable by a drive mechanism. The conveyer is capable of bi-directional movement of labware, such as microplates, between integrated instruments under control of the main process controller of FIG. 2.

Track elements 7, 13 may be connected to each other and/or laboratory equipment using a connector 2 illustrated in FIG. 1c. Furthermore, connections may be made between two track elements so that a track may be lengthened and the two connected tracks function as one unit. When the robotic arm 3 delivers a magazine or growth dish to the track system 7, the magazine or growth dish 5 may be transported to a colony picking robot 9 via a track element 7.

A colony picking robot or colony picker element 9 generally receives a growth dish 5 from the track system element 7 and picks a bacterial colony from the solid medium surface. The colony picker 9 is capable of locating and picking bacterial colonies from the solid medium. surface and inoculating a liquid medium-filled well of a microplate. The microplate has distinct and separate wells, such as a 96-well deep-well culture microplate, or any other microplates such as a 384 and 1536-well microplate. The individual wells enable the picked bacterial colonies to grow separately without contaminating neighboring growing colonies. The colony picking robot 9 generally contains its own control unit and may be set up and calibrated so that the machine is capable of recognizing where plates are located and the movements that must be accomplished for the successful picking and inoculation of a microplate.

While the colony picking element 9 is in operation, culture microplates may be introduced into the system via similar magazines from a plate stacker 17 and illustrated in FIG. 1d. One such plate stacker element 17 is made by Hudson Control group called StackLink and may be used for storage of lab ware, and then introducing lab ware such as microplates to the automated proteomics workcell 1. The StackLink by Hudson Control Group may also be expanded using the StackLink expansions 17-1 illustrated in FIG. 1e to increase capacity of the workcell 1. StackLinks may also be connected to each other. The stacker element 17 has an active mechanism for pickup and release of lab ware. The stacker element 17 has two stacks each with a capacity of 30 standard microplates, or 60 if the expansions are used. Additionally, the stacker element 17 is configured with its own controlling device to enable proper performance. For example, the controlling device allows for the pickup and release of lab ware in addition to bi-directional control to allow plate shuffling and random access. Although only two plate stacker elements are illustrated within the embodiment of the workcell 1 herein described, multiple plate stacker elements may be configured in one workcell 1 system to allow for an increased total capacity.

In the preferred embodiment, a plate stacker element 17 feeds the culture microplates onto track elements 7, 13 for transport. Depending upon the origin of the microplate, the robotic arm 3 may need to be utilized to position the microplate on the correct track element 13 so that the microplate may be transported to an automated liquid dispenser element 11. The automated liquid dispenser 11 also contains its own control unit. The control unit may be programmed to dispense specific amounts and types of liquids. For example, the automated liquid dispenser element 11 may fill in a sterile fashion the wells in the microplate with a desired quantity of liquid medium for growing cells. The medium-filled culture microplate may then be transported along the track element 13 to a position 23 where the robot arm 3 can lift and move the plate onto a second track element 7 for transport to the colony picking robot 9 or to a liquid-handling/pipetting robot 15.

The colony picking robot 9 is connected by the same track element 7 to other instruments and microplate storage and feeding devices such as a plate stacker element 17. Microplates and growth dishes move into and out of the colony picker on track element 7, as described above, to enable them to be located within the colony picking robot's working area. The colony picking robot element 9 has its own software and controlling mechanism, but when integrated into the workcell it may be controlled by the workcell controller or main process controller illustrated in FIG. 2. The colony picking robot 9 performs a variety of tasks related to transferring bacterial (or other types of microbial) colonies, growing on dishes delivered to it, into medium-filled wells of microplates likewise delivered to it.

Frequently, there is a need to deliver the microplate to a liquid handling/pipetting robot 15 to inoculate some of the wells in a microplate with bacterial colonies of known characteristics to serve as controls for comparison to the colonies of unknown characteristics growing on the growth dishes. This necessitates an automated transfer of the microplate(s) from the liquid-handling/pipetting robot 15 to the colony picking robot 9. This automated transfer is accomplished by a track element 7 or alternatively by a robotic arm 3. The microplate may also be moved from the colony picking robot 9 to the liquid-handling/pipetting robot 15 immediately following inoculation of growth dish(s) to add controls, or possibly other materials required to be in the microplate following inoculation. The track element 7 that delivers microplates to the colony picking robot 9 may be used, in one embodiment of the invention, to move microplates between the liquid-handling robot 15 and the colony picking robot 9. Alternatively, a robot arm 3 could also provide the means to automatically transport microplates between the colony picking robot 9 and the liquid-handling/pipetting robot 15.

When the colony picking robot 9 has finished performing its programmed job, such as a transfer between the growth dish and the microplate, hereinafter referred to as a culture plate, the old plates need to be removed. old dishes or culture plates, or both, may then be transported out of the colony picking robot's working area via the track element 7 or alternatively a robotic arm 3 to a holding location in the system such as a plate stacker element 17 where they may be manually removed by a user. Alternatively, old plates or dishes may be stored at stacker elements 17 temporarily and then moved to the next piece of equipment if further experimentation using those plates is needed.

When a culture plate is finished being inoculated by the colony picker element 9 it is typically directed toward an instrument or location where it may be incubated. An incubator 19 provides automated control over the proper temperature and environmental conditions as to enable the newly inoculated bacterial cells to grow. The incubator 19 is automatic and may contain its own control unit which has been integrated into the workcell and may be controlled by the main processor controller of FIG. 2. The incubator 19 is capable of incubating more than one microplate at a time. This allows microplates to be loaded into the incubator in a repetitive manner after each plate has undergone processing from the colony picker element 9 and incubation preparation.

The physical connection between the colony picking robot 9 and an automated incubator 19 in the example is unique to this system 1. Incubator elements 19 may be adapted for use with an automated proteomics workcell 1 by using an intermediate device to connect the incubator 19 and the track element 13 or robotic arm 3. One such device that may be used for this is the DirectLink adaptor from Hudson Control Group illustrated in FIG. 1f. Another embodiment of the means of transporting culture plates from the colony picking robot 9 is a means, such as a robot arm 3, to remove the culture plate from the track element 7 servicing the colony picking robot 9, and place it onto a track element 13 which may deliver it directly into an automated incubator instrument 19. This connection via a robot arm 3 enables the overall system to remain compact and more efficient. An extension of the colony picking robot's track system to reach the incubator directly would be another viable embodiment or using the robot arm without track units.

Before moving the culture plate to the incubator 19, the system may move the plate to ancillary instruments in order to properly prepare the plate for incubation. One such instrument may be an automated plate sealer element 21. The plate sealer 21, may adhere a semi-permeable membrane to the top surface of the culture plate to allow gas exchange during incubation while protecting the wells of the plate from microbial contamination. The ability to incorporate such ancillary instruments into the plate transport system gives the system practical diversity of implementation. Each ancillary instrument including the plate sealer 21 is controlled individually by their own respective controllers that program the machine and communicate the activation and results of the desired operations with the main controller of FIG. 2. Delivery of plates to ancillary instruments may be by robotic arm 3 or track elements 7, 13 or both.

During each culture plate's incubation period, other culture plates may be produced in a similar manner and added to the incubator 19 in a like manner, to form a sequential series of inoculated culture plates being simultaneously incubated to produce economically viable quantities and varieties of colony growth to facilitate the desired research effort.

When each culture plate's incubation period has concluded, the automated incubator 19 may deliver each plate back onto the track element 13 for transport to its next operation, which is usually, though not always, the extraction and purification of the numerous DNA-insert containing plasmids from the bacterial cells located in each of the culture plate wells. This process is often called plasmid preparation. The plasmid preparation may proceed even while the other culture plates continue their own, later-started incubation periods.

One embodiment for automatically moving culture plates-from an automated incubator 19 to a plasmid preparation workstation, which may be a liquid-handling/pipetting robot 15 or a dedicated set of instruments organized to perform the plasmid preparation process, would be to move the plate along a track element 13 to a pickup position 23 where a robot arm 3 would transfer it to a second track element 7 that would deliver it to the plasmid preparation workstation or liquid handling/pipetting robot 15. A pickup position 23 for a robotic arm may include a sensor 24 coupled with a stop positioning device 26 that will stop a plate on the track element 7 so that it may be picked up by a robotic arm 3. The sensor may be a switch that detects the presence of a plate and sends a signal to a controller of the track system and/or the main process controller. An example of a sensor of this type is the plate sensor 24 from Hudson Control Group illustrated in FIG. 1g. An example of a stop positioning device 26 is also illustrated. Alternatively, the robotic workcell may be configured to allow the plate to move on the track elements 7, 13 directly to the plasimid preparation workstation or liquid handling/pipetting robot 15.

The plasmid preparation workstation 15 generally, extracts plasmids contained in the growing bacteria using techniques well-known to those skilled in the art. The plasmid preparation workstation 15 may then be used to purify the extracted plasmids. This is also performed using techniques well-known to those skilled in the art. Plasmids may then be placed into a second microplate, so that upon the completion of the plasmid preparation, the plasmids exist in a purified state in a secondary microplate whose individual wells contain just those plasmids from a single source well of the culture plate. The secondary plate may be delivered to a magazine such as a plate stacker element 17 for storage via a track element 7 or alternatively via a robotic arm 3. There they eventually might be removed from the system manually. In the alternative, as in the embodiment of the system shown here, the secondary plate containing the purified plasmids may be further processed by the robotic workcell system 1. This often involves transport of the plate containing the purified plasmids to the liquid dispenser 11 via the track elements 7, 13, the robotic arm or both, so that the automated addition of reagents and other materials may occur. Generally, this is done to remove DNA inserts from their plasmid library setting in bacteria and directionally clone them into secondary plasmids. The secondary plasmids are placed into a new bacterial, yeast, plant or animal cell expression plasmid and grown in the appropriate medium. This procedure is intended to promote the production of protein material from the DNA inserts, a process know as gene expression.

The robotic workcell 1 facilitates expression either while still in solution, called in vitro expression, or by transforming or transfecting the secondary plasmids into prokaryotic or eukaryotic cells for an additional incubation and growth to produce protein within those cells, called in vivo expression. To perform the transformation or transfection into the prokaryotic or eukaryotic cells, the microplate containing the secondary plasmids may be transferred to additional instruments, via the two track elements 7, 13 and robotic arm 3, to allow the proteins to fold into the proper functional configuration.

When the incubation period is finished, the microplate may be delivered, in this embodiment, onto the track system 13 servicing the incubator 19, then transferred to a series of instruments generally including a liquid handling/pipeting robot via the tracks elements 7, 13 and the robotic arm 3. The instruments generally would add required reagents or materials to permit a detecting device like a plate reader 25 to measure the levels of expressed proteins. The expressed proteins are analyzed for function (binding and activity). These operations form the basis for plasmid-based functional proteomics.

After the proper reagents and materials have been added by the instruments, the system automatically transfers the microplate to a detection device, via the track elements 7, 13, robotic arm 3 or both to a detection device frequently called a plate reader 25. A plate reader element 25 may determine concentrations in solution by light refractive, absorbance, luminescence, fluorescence properties or by molecular mass, such as by using a mass spectrometer device. The plate reader element 25 generally automatically measures the protein or DNA production in each well. The results are recorded or archived in the form of a computerized database or data file for later analysis by the researcher. Such data of the database may also include a complete history of a sample concerning the processes performed on the sample with the instruments of the workcell as recorded and tracked by the system.

In a preferred embodiment of the invention, one or more reader elements 27 such as bar code readers are utilized. The reader elements 27 are preferably placed on or around the track element 7 leading to the colony picker robot 9, and on or around the track element 13 leading to the robotic incubator 19. The reader element 27 may be used to tag and keep track of where individual samples are located at any time point during the operation of the automated proteomics workcell 1. For example, all growth plates, magazines, or culture microplates may be tagged with labels that are capable of being detected by the reader element 27. Prior to any growth plate, magazine, or culture microplate entering the colony picker 9 via the track element 7, it may be scanned by the reader element 27. The reader element 27 is connected to a computer that monitors and keeps track of the samples and where samples are located at any given time. The reader element 27 located near the automatic incubator 19 serves a similar purpose and allows for tracking of samples during the experimental process. Alternatively, different scanning and tracking systems may be used.

Preferably, software controlling the workcell 1 has the capability of tracking bar code information using both 1D and 2D style bar code tracking. The software also has the ability to schedule automated plate movements. The software is capable of capturing and storing data collected from a bar code reader element 27 on and off the workcell. A printed report may be generated as well as hard drive storage of plate movements and data obtained concerning functions performed on those plates.

In one embodiment, a print and apply device or alternatively, a bar code print and apply device (both marketed by Hudson Control Group) may be integrated to work with the proteomics workcell. The print and apply system is an integrated platform designed to be installed in any laboratory. This system is generally a high performance, high capacity solution for bar code labeling of microplates. The system generally consists of a Computype LAP-4100H 600 dpi Printer—applicator and a Hudson PlateCrane™ microplate handling robot. The system is capable of applying labels to any or all sides of a microplate. The 600 dpi printer provides the resolution needed for reproducible printing on labels sized for microplates. The capability to use labels up to 3.3″ wide allows use of longer bar codes and the printing of human readable text next to the bar code. Each print and apply system may be individually tailored to a workcell as necessary. In an integrated proteomics system, with the print and apply device, a common robot such as the Plate Crane may be utilized rather than supplying multiple robotic arms.

The use of a two track element system in the illustrated embodiment of the present invention in FIG. 1 facilitates a more compact layout and more efficient plate movement, but is not the only configuration that could accomplish successful automation of laboratory experiments. One alternative might include having one track element that serves all laboratory instruments and machines that have been integrated into the robotic workcell 1. For example, the incubator 19 and the plasmid preparation workstation 15 might be connected via the same track element 7. This would enable the plate to be brought between these machines on one track element. Alternatively, a robot arm 3 could be used to maneuver plates between all equipment contained in the robotic workcell 1.

In this respect the robotic workcell 1 allows for the flexibility to arrange the integrated equipment in different locations. This provides an advantage because the robotic workcell 1 may then be conformed or built to the specifications of a room or laboratory location and configuration. Hence, it is capable of being built virtually anywhere in the laboratory. The flexibility is accomplished by using different combinations of track elements 7, 13 and/or robotic arms 3 so that microplates may be moved between laboratory equipment and machines in accordance with the user/researchers desires. For example, each piece of equipment used in the robotic workcell 1 acts as a building-block and each building-block comes together to form the entire workcell 1 system.

The ability to change the arrangement also allows for flexibility in changing which machines are included in the robotic workcell. For example, in the preferred embodiment a thermal cycler 29 is included in the robotic workcell. A thermal cycler 29 could be a PCR machine which is used to replicate DNA or RNA to make hundreds of copies. Also in the preferred embodiment a centrifuge machine 31 is included in the robotic workcell 1. A centrifuge machine 31 spins samples at high speeds in a circular motion in order to separate components by weight. Both the centrifuge machine 31 and the thermal cycler 29 may be accessed either by track element 7, 13 or by the robotic arm 3 or by a plate manipulator that is part of the liquid handling/pipetting robot. Some laboratories may not have the need for specific equipment or machines contained in the preferred embodiment of the robotic workcell 1. In that case, the robotic workcell may be built with fewer components than those mentioned.

The building-block approach of the workcell 1 system means that only the imagination limits the potential uses to which it may be applied. For example, as illustrated in FIG. 1h, the robotic arm. 3 may be implemented to pick up a plate from a track element 7 rotate it 90 degrees, and place it on a perpendicular track element 13. This allows more control and better use of bench space when expanding systems or designing them to fit within a designated area.

Many processes such as plate replication, plate consolidation, and hit-picking require different types of plates to be supplied to the liquid handler 11 (mother and daughter or source and destination). The expandability of the workcell 1 makes it easy to add more capacity for different plate types by adding or removing plate stacker elements 17.

The flexibility of configuration allows the workcell 1 to be built with much a lower investment than for systems based on rail mounted multi-axis articulating robotic arms 3. Furthermore, the rapid pace of development in drug discovery can mean that today's assay needs to be eliminated in favor of a newly developed system. Since the workcell 1 is easily reconfigurable, a new system may be quickly built, providing a greater return on investment. As illustrated in FIGS. 1i and 1j the ability of the workcell 1 to be reconfigured may be seen. FIG. 1i shows a workcell that has been reconfigured in FIG. 1j. One washer is removed and the plate reader element 25 is replaced with a different and upgraded model. A reconfiguration such as this is easily accomplished in comparison to more permanently installed large scale systems.

Additionally, various plate processing steps often take place in a series of “offline” steps. For example, a group of plates may be processed on a washer, and then placed on a liquid handler 15 for the next step(s). The workcell 1 system makes it easy to connect these processes together to further increase walk away automation. Multiple processes may be automated without the need for articulating robotic arms mounted on linear rails. For example, as illustrated in FIG. 1k the workcell 1 may have a washer element 11 and liquid handler element 15 linked together to build a system that will automate the washing and liquid handling of a series of plates.

The capacity of a workcell system may easily be increased by adding additional plate stacker elements 17 or adding StackLink expansions 17-1 to a plate stacker element 17. Each plate stacker 17 has two stacks which are independently addressable. If one is used, one stack will be the “input”; this is where the plates to be processed will be initially loaded. The second stack will be the “output”. After the plates are processed, they will be returned to this stack. If a second plate stacker element 17 is added to the system, then both stacks on one plate stacker element 17 may be used as inputs, and both stacks on the other plate stacker element 17 may be used as outputs.

Many microplate-based instruments, such as reading/imaging systems, do not have physical designs that allow track-based feeding of plates. These instruments are easily integrated into a workcell 1 system by using a robotic arm 3. The robotic arm 3 may be a simple “pick-and-place” robot arm. It differs from complex articulating robotic arms because its sole function is to move a plate from one fixed location to a second fixed location. This allows a simpler design which in turn is easier to configure and is more reliable. The robotic arm 3 allows additional instruments to be combined so that automation of multiple assay steps may occur or even complete room-temperature assays may be performed. Alternatively, the robot arm may be a cylindrical robot arm, such as the PlateCrane from Hudson Control Group, or other robot arm configurable for automated transfer of sample plates or microplates between multiple locations.

Plate-storage systems such as incubators or medium-scale freezers are available in “automation-friendly” formats that allow robotic access to a plate for entry or exit. The robotic arm 3 may be used to move plates into and out of the incubator element 19. A system can be built to automate an entire assay. By way of example only, the workcell 1 may be quickly configured with all of the instruments required for a full assay. In this case the plate stacker elements 17 would serve as the original source of plates and a removal source of plates. Plates would be sent to the primary washer, liquid handler 15, and secondary washer as required by the assay. Bi-directional control of a plate via track elements 7, 13 or robotic arm 3 movement allows plates to be brought between washers and the liquid handler 15 for additional reagent dispensing steps. After performing initial liquid handling, the plates are placed in the incubator for incubation. After the appropriate incubation time, which is specific depending upon the experiment, the plates are sent to the reader via the track elements 7, 13, robotic arm or both.

The workcell 1 may be capable of automation of a single instrument for walk away automation. For example, a common activity is the processing of a group of microplates with a single instrument, such as a plate washer. When performed manually, the user must load each plate one at a time, activate the washer, wait 1-5 minutes while the plate is washed, remove the plate, and repeat the process. Because the processing time of the washer is short, there is not time for the user to begin other work or perform other tasks.

Processing a group of 60 plates in this manner requires several hours of work. For this process, a basic workcell may be designed to automate plate washing for a batch of plates as illustrated in FIG. 1k. The user simply loads 60 plates in a plate stacker element 17, and starts the system. The system of this type will feed plates from the input stack 17a to the washer element 11, activate the washer element 11 and return the plates to the output stack 17b. The process still will take a few hours (depending on the washer cycle time), but the operator is free to walk away to perform other more productive tasks while the plates are processed. Moreover, such a process may be integrated into an automated proteomics system as discussed herein.

It is also recognized that other laboratory equipment and machines not mentioned herein may be capable of being incorporated into the robotic workcell 1. These machines and equipment may be incorporated through the same track element system 7, 13 and/or robotic arm 3. It is also recognized that those skilled in the art will cater the usage of the robotic workcell 1 for their particular purpose. For example, the order of the events explained above may occur in any order depending upon the type of experiment or work being conducted. In a preferred embodiment of the invention, the entire system is controlled by scheduling software which permits the simultaneous operation of the various instruments in the system as well as the simultaneous movement of labware between the various instruments. Thus, such a control system may be programmed with different schedule processes as de scribed using the automated instruments integrated into the proteomics workcell.

The operation and control of the robotic workcell 1 is generally illustrated in FIG. 2. Software permitting such operation is contained inside a computer 36 which operates as a controller for the entire system. The software may use an icon-based drag and drop method for creation of a laboratory process. Comprehensive menu control icons allow for simple or complex methods or processes to be written for the system. Multiple method threads may be written and executed simultaneously. The software has simple “VCR”-type buttons for starting and stopping methods. Each automated instrument installed in the workcell has an icon that allows for the configuration of each such component. Depending on the process, the user may configure the workcell 1 by programming the individual components so that a specific experiment may be properly conducted.

For example, each instrument or component 53 has software or firmware that may be actuated by the scheduling software. Software such as SoftLinx™ offered by Hudson Control Systems is loaded on the computer 36 and communicates with each individual component's 53 software or firmware in a manner specified by the component's vendor/manufacturer so that the component's activity may be controlled at the appropriate time in each process.

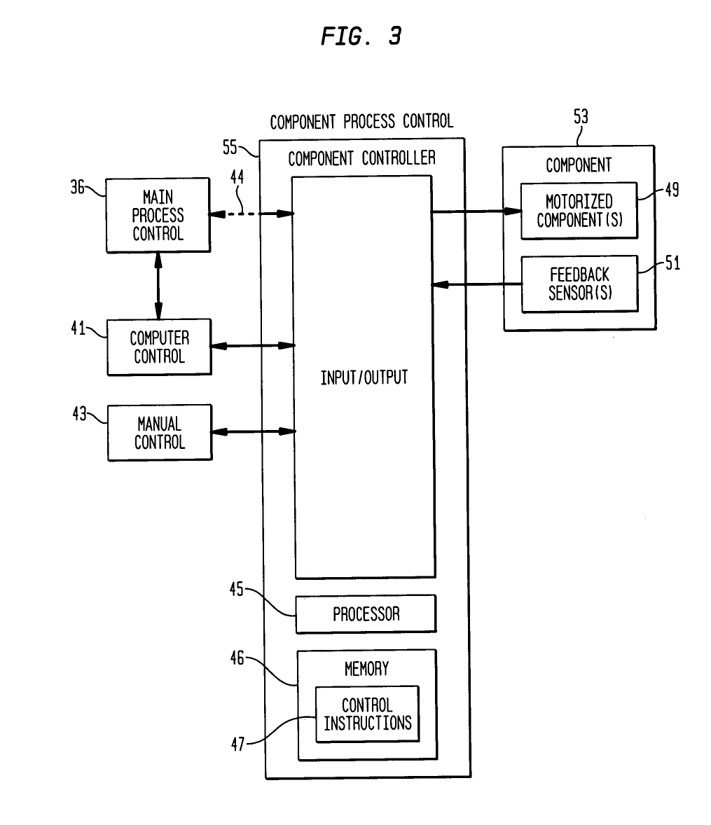

The scheduling software communicates with component's firmware/software and ultimately controls the functions of the workcell 1. This integrated communication is illustrated in FIGS. 2 and 3. In FIG. 2 the computer 36 that contains scheduling software generally has a processor 37 and memory 38 and is capable of input and output controlled by the processor 37. The computer 36 is coupled to a plurality of components 53 that are integrated to make up the workcell 1. The computer 36 acts by outputting commands to individual components 53 of the robotic workcell 1. For example, the individualized components 53 of the robotic workcell 1 may include a robotic arm element 3, track element(s) 7, 13, colony picker element 9, liquid dispenser element 11, pipetting robot element 15, plate stacker element 17, robotic incubator element 19, plate sealer element 21, plate reader element 25, barcode reader element 27, thermal cycler element 29, and centrifuge element 31 or any combinations thereof as previously discussed. All components 53 have had firmware/software and communication software so that the components are capable of receiving and sending commands, messages and or data to and from the main process control computer 36.

The scheduling software contained on the computer 36 creates the control instructions 39, which are then stored in the computer's 36 memory 38. The control instructions 39 may be modified according to the user's preferences. For example, the user may choose which pieces of equipment need to operate, and the order of operation so that a specific experiment may be performed. The control instructions 39 stored in the memory 38 are processed by the processor 37 and communicated to the specific component 53 of the robotic workcell 1 that needs to function at a particular time.

Illustrated in FIGS. 2 and 3 are the way the controllers of the workcell 1 and components 53 communicate. Each component 53 is capable of communication with the processor 37 through the component's 53 own controller 55. The computer 36 sends a signal to the component computer control 41 to perform the programmed function and then waits for a return command from the component computer control 41 that the function has been completed. The completion signal may be sent by the component control computer 41 when the component 53 is done operating. The signal sent back to the computer 36 is received by the processor 37 shown in FIG. 2. The processor 37 then sends the next set of control instructions 39 from the memory 38, which is directed towards operation of a different component 53 of the robotic workcell 1. By this process, plates are transferred between the different instruments for automated control of a proteomics workcell.

Referring to FIG. 3 the general control of an instrument or component 53 is illustrated. The component represented in FIG. 3 may be any integrated component of the workcell 1 and not just those illustrated in FIG. 2. The main process control computer 36 sends a signal to the computer control 41 of a specific component 53 in the robotic workcell 1. The computer control 41 may be the software (or firmware) supplied with the component 53 that has been modified and made capable of being actuated by the signal from the main process control computer 36. The computer control 41 operates the component controller 55 which generally contains the ability to input and output signals, a processor 45, and memory 46 for control instructions 47. The component controller 55 is coupled to the motorized part(s) 49 on the component 53 contained within the robotic workcell 1. The component 53 preferably also contains feedback from sensors 51 for closed loop feedback to the processor 45 contained in the component controller 55. Therefore, each component integrated into the workcell 1 has a component controller and optionally a computer control 41. Optionally, open control may be implemented.

Each component 53 may also be controlled via individual control 43. This allows for individual function of a particular component. For example, if a researcher in a laboratory only wants to run a PCR reaction in the thermal cycler 29, samples can be put into the thermal cycler 29 manually and then turned on and set to run independently from being controlled by scheduling software of the main controls. Other components within the robotic workcell 1 are also capable of manual and individual function in the same manner.

In an alternative embodiment, the main process control computer 36 may control the component controller 55 directly. This is indicated by the dashed line 44 and may act to make the process more efficient. The system would still be capable of closed loop feedback. In this embodiment the component controller 55 will act as both the computer control 41 and the component controller 55.

The processor 37 of the main process control computer 36 shown in FIG. 2, outputs a signal to the component computer control 41, shown in FIG. 3, based on the control instructions 39 stored in the main process control computer's memory 38. The user controls the control instructions 39 based on the programmed process created with the scheduling software. The component computer control 41 inputs control instructions 47 to the component controller 55 which are stored in memory 46. The processor 45 of the component controller 55 directs the input/output of the control instructions 47 to the motorized part(s) 49 of the component 53. The feedback or sensor 51 inputs a signal back to the component controller 55 indicating completeness of the desired function. The processor 45 directs the output of a signal back to the component computer control 41, which directs a signal of completeness of the desired function to the main processor controller 36 shown in FIG. 2. The processor 37 of the main processor controller 36 receives or accesses a new set of control instructions 39 from the main processor controller memory and another process is performed with the next component used in a desired testing procedure.

The above is repeated until all control instructions 39 from the main process control computer have been completed.

Additionally, when the main process control computer 36 has received information from a component 53, it may store information into memory 38. For example, data from the bar code reader element or data results from the individual pieces of equipment such as the plate reader element 25 may be stored into memory 38. The information that is stored in the memory 38 may then be placed into a database and organized so that the researcher may read and interpret the results of specific tests and/or track the progress of the experiment being performed. The database may then produce a printed report of all movements and data obtained, as well as storing the information in the memory 38 of the computer 36.

The current invention is capable of maximizing throughput and efficiency of an automated laboratory. In order to maximize throughput and efficiency of laboratory operation, the robotic workcell 1 allows for a researcher to be preparing culture plates, while all the automated experiments are being performed by the robotic workcell 1. For example, the robotic workcell 1 might be performing several tasks at once. Some plates may be incubating, while others are undergoing the plasmid preparation process, and still others are in varying stages of the in vitro or in vivo protein expression process.

The above will be repeated until all equipment programmed by the scheduling software scheduled to run are complete. The software from the computer 36 controls the movement of the lab ware between the laboratory components 53 via the track element system 7, 13 and the robotic arm element 3. Additionally, in the preferred embodiment, the computer 36 may receive information from the bar code reader element 27, or any of the third party components 53, which can then be stored in a data collection part of the computer 33 and may be capable of automatically being placed into a database.

Additionally, the main control processing computer 36 may be set up so that it may communicate with a cellular phone and/or pager. Upon completion of an experiment run the main control processing computer 36 may notify the user by calling a cellular phone and/or pager. This notification system may also be utilized by the control processing computer 36 upon an error condition. For example, if something goes wrong with the process or the robotic workcell 1 malfunctions the control processing computer may notify the user via the paging system so that the user may come and fix the machine or attend to the problem.

In an alternative embodiment, as illustrated in FIG. 2, the main process control computer 36 that controls the automated process of the robotic workcell 1 may be connected to another computer 33 that controls the main process control computer 36. The computer 33 may be connected to control a series of automated processes or multiple robotic work cells 1. For example, a computer 33 may be set up as a main control center that controls more than one workcell directed towards different laboratory processes. Communication between the computer 33 and the main process control computers 36 of each individual workcell 1 occurs via communications with the processor of the computer 33.

Preferably the scheduling software is multi-threaded that may run multiple instruments simultaneously, route data to appropriate servers, and provide status information to users through databases or other forms. The scheduler employs an easy to-use “drag and drop” method builder that enables a researcher to modify any system process and/or develop their own process that will be performed by the workcell 1. Preferably the scheduler is designed to make the programming and operation of lab automation workcells easy for routine operation by lab personnel and flexible for custom modification by programmers.

The scheduling software allows for multitasking executable programs with built-in dynamic scheduling. Therefore, many programs or pieces of equipment may be operated at one time. For example, connectivity may exceed 100 lab automation devices that have interfaces capable of being adapted for work with this software and integration into a workcell 1. Additionally, the program has scheduling features that permit equipment to be operated or used for tests at a specific time set by the user on a regular basis or a one time event.

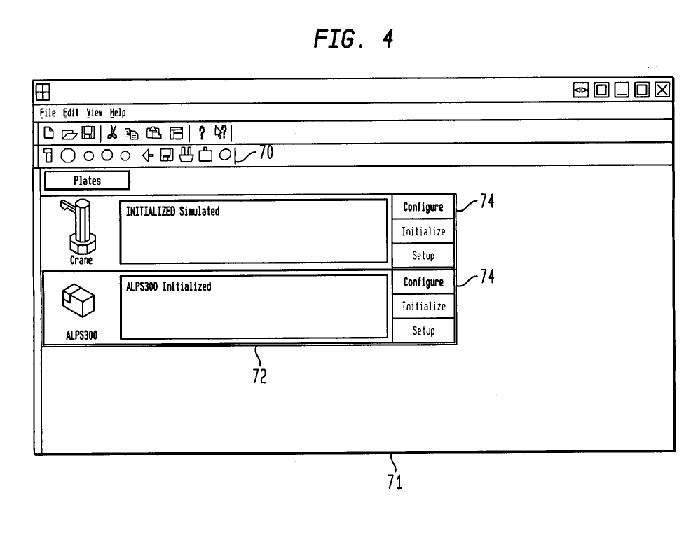

One aspect of the scheduling software is the configuration interface. This screen is illustrated in FIG. 4. It is used to set up the workcell 1, that is, the components that will be used in a specific run of the workcell 1 may be selected. Icons for installed instruments appear on the toolbar 70. When the programmer wishes a specific instrument to be used in a specific run of the workcell 1, the icon on the toolbar 70 is dragged and dropped to the lower half of the screen, which is the system configuration screen 72. Appearing in the system configuration screen 72 are all components that the user has added and are needed to run the desired experiment. Once a component has been dragged from the toolbar 70 to the system configuration screen 72 a special configuration button 74 appears for each specific component added to the system configuration screen 72 which allows for further configuration of the individual component.

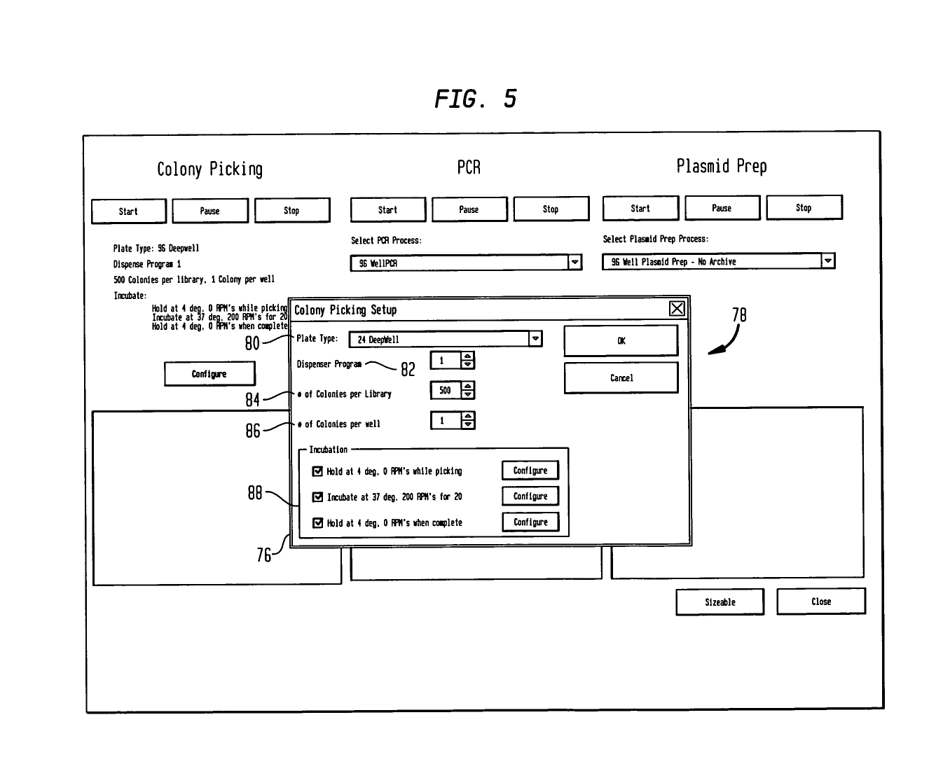

When the configuration button 74 is pressed the configuration screen for the specific component is selected. The configuration screen is illustrated in FIG. 5. By way of example only, the foreground screen 76 displays the configuration/setup for the colony picking machine. The colony picking machine is one of the machines that has been chosen for use during the current experiment, as can be seen by the background screen 78. The colony picking setup/configuration screen allows for configuration of properties such as the plate type 80, dispenser program 82, number of colonies per library 84, colonies per well 86, and incubation properties 88 for storage before, after and during colony picking. Overall, this configuration of the individual component allows for basic setup of the main parameters that will be used in the run of the workcell 1.

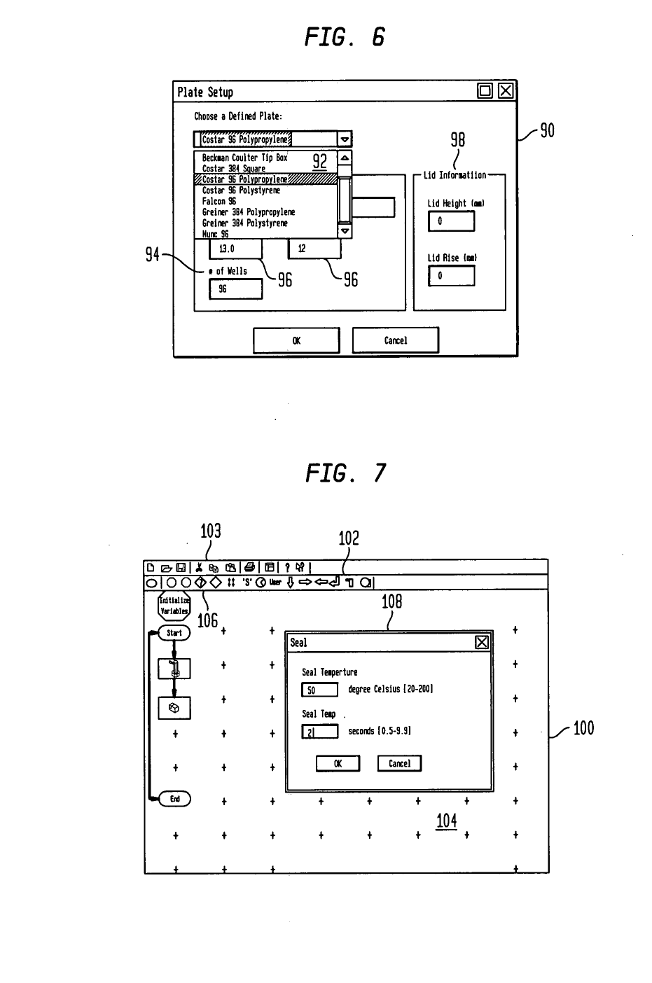

Another aspect of the scheduling software is illustrated in FIG. 6 and is the plate setup screen 90. The plate setup screen 90 offers a preprogrammed list of plate definitions contained in a drop down menu 92. The user/programmer may choose from one of the plate definitions contained in the drop down menu 92 or the user may enter a new plate definition by entering the parameters in the respective boxes below. For example, as can be seen in the background to the drop down menu 92 the information with the number of wells may be entered along with plate dimensions 96 and lid information 98. The user/programmer may save the custom plate information for use during a further run.

FIG. 7 illustrates the method or process editor screen 100. This screen is used to develop instructions for processing experiments with the system and uses the drag and drop interface similar to that of the configuration screen. Icons for installed interfaces 102 appear on the toolbar. The user drags the icon 102 needed onto the method editor screen 104. Once icons for installed interfaces 102 have been placed appropriately on the method editor screen 104 the method logic icons 106 may be used to custom configure the movement of samples through the entire workcell 1 and to change around parameters to individual interfaces. The example shown in FIG. 7 is the seal parameters box 108. As seen in the illustration seal temperature and seal time may be altered to desired parameters.

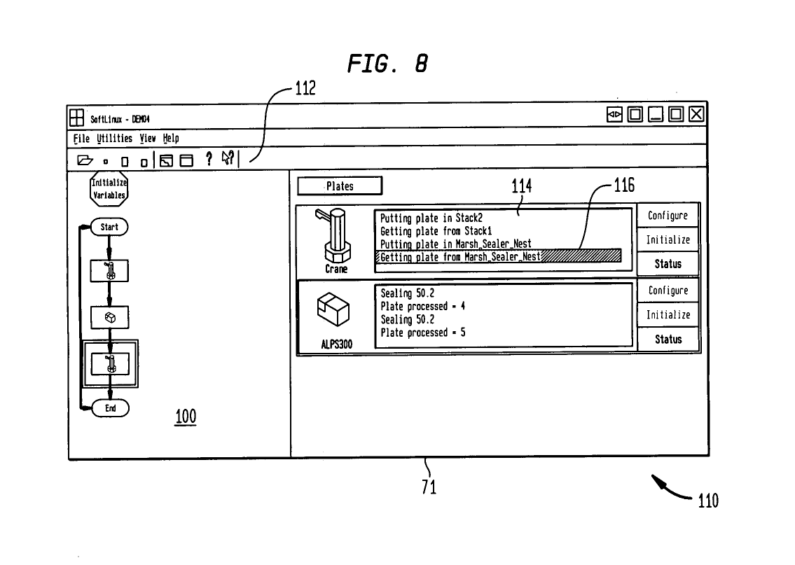

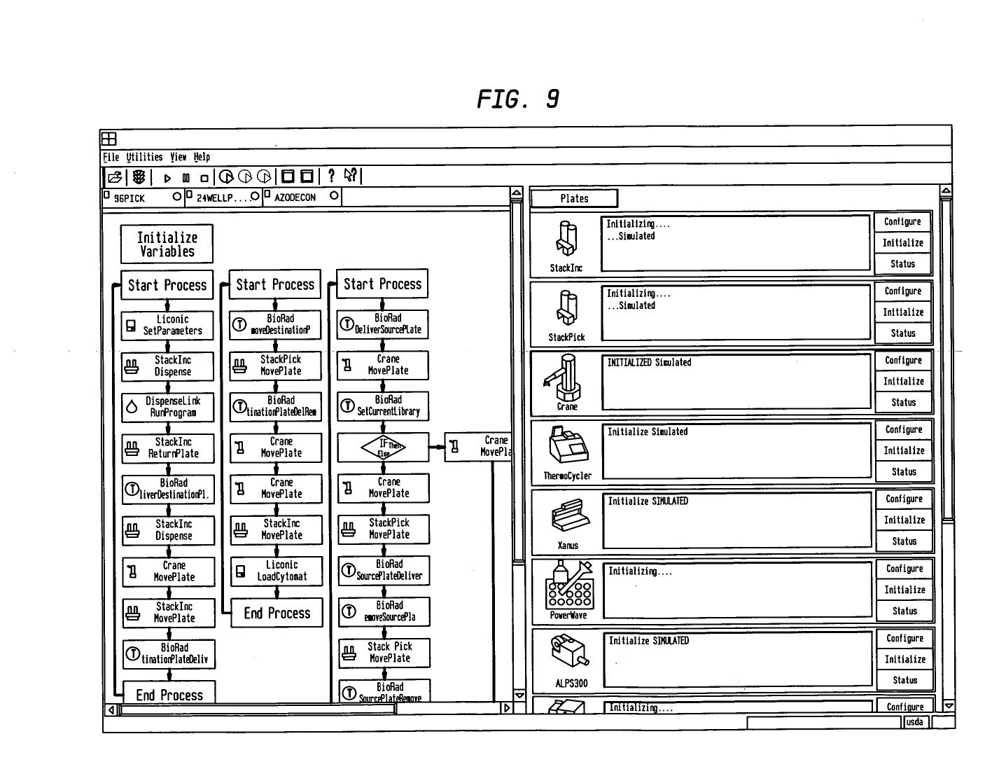

FIG. 8 illustrates the method run screen 110. This screen is used to start methods with simple VCR-style control buttons which can be seen on the tool bar 112 at the top of the screen. The screen is a split screen with the left half of the screen showing the method display screen 100 and the right half showing the configuration screen 71. On the method display side of the screen 100, the actual method that is to be performed is shown. On the right side of the screen the configuration screen 71, which shows the instruments configured in the system, contains a box 114, next to each component. Inside the box 114 running steps or automation steps for each instrument are shown. The current event being performed by a particular automated instrument appears highlighted 116. The example screen interface illustrated in FIG. 8 of the method run screen 110 illustrates only use of the crane and plate sealer element 21. However, the workcell 1 may be configured in very complex ways as shown in FIG. 9. FIG. 9 is the method run screen shot with a complex process configured to run the workcell 1.

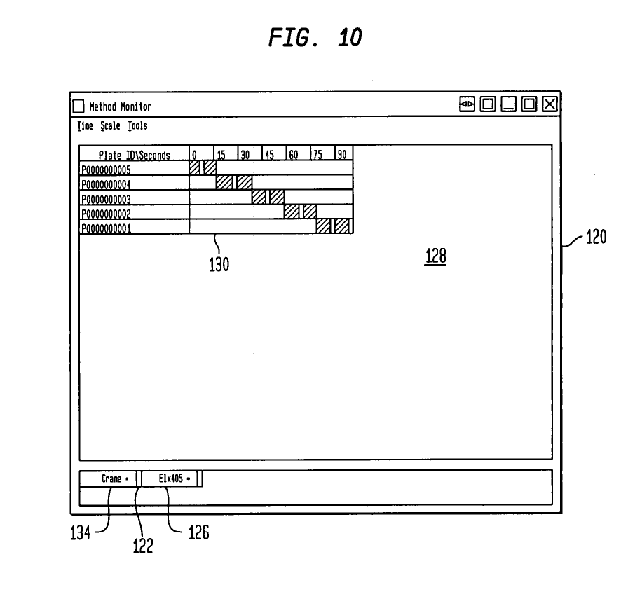

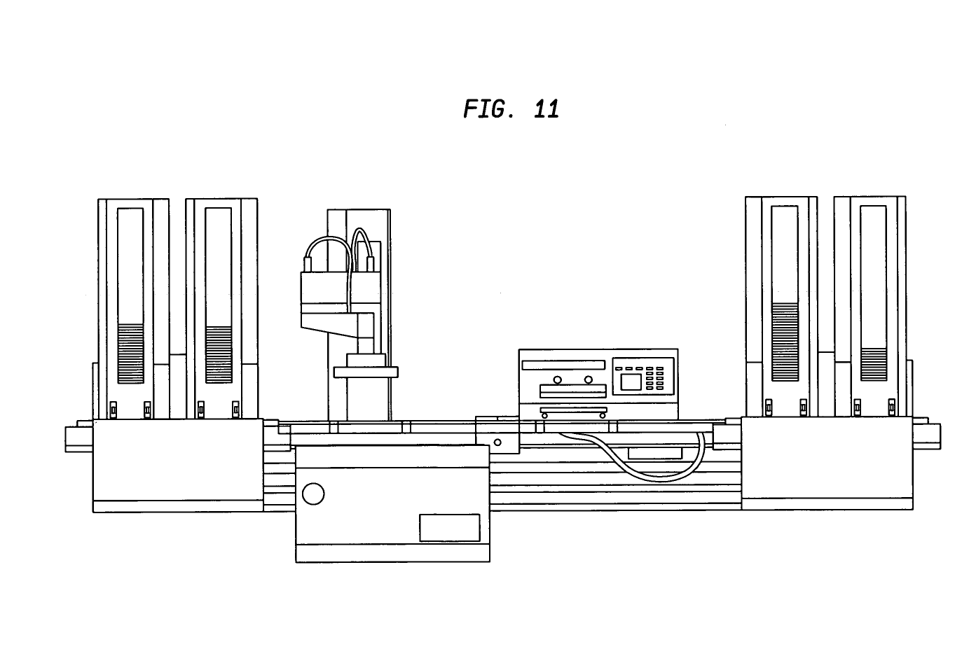

Illustrated in FIG. 10 is the predictive scheduler display 120. This screen provides a graphical display of plate movement and timing for each device in the workcell. This illustrated interface is an example showing the robotic arm/crane 3 and plate sealer element 21. This is shown at the bottom of the screen shot and in this instance there are two buttons 122 which indicate the two different interfaces (crane and sealer). When the crane button 124 is selected the upper portion of the screen 128 displays the movement of each plate with respect to the crane interface. When the sealer button 126 is selected the upper portion of the screen 128 displays plate movement with respect to the sealer. The current predictive scheduler display 120 shows when each plate has been handled by the crane 130. The screen also assists in method optimization. For example, if the user/programmer believes the timing of the experiment to be incorrect after viewing the predictive scheduler they may go back to a previous screen to adjust the experiment process and correct the potential problems.

The scheduler software also contains dynamic scheduling and advanced method functions. It uses event-driven dynamic scheduling to ensure the most efficient plate movement and the most efficient use of the automation robotics. At run time, the software can automatically use the integrated robotics to move plates to the next available instrument in the workcell, within the rules of the method. Complex testing processes can be built using functions such as loops, if-then statements, math and string functions, and timers for incubations. The software can automatically manage a process that includes incubations by keeping track of each plate as it is being processed.

For configuring use of each component 53 each instrument preferably includes its own configuration data to be reconfigured as needed. For example, the scheduler includes a “New Interface” function that will open a new interface file on a template that easily allows creation of custom configuration by end users.

Generally, the present invention is useful when performing genetic research. For example, the invention may apply to plasmid preparation, restriction mapping, in vitro protein expression, sample assay, and mass transformation of prokaryotic, fungal and yeast cells, and/or transfection of eukaryotic cells. Therefore, the invention is an integration of the necessary automated equipment into a workcell 1 that allows for a continuous experimentation from library clone set maintained in bacteria to the final plasmid preparation used for the chosen type of protein expression (in vitro or in vivo), then to the final assay in a production manner. However, the invention may be adapted for use with other research purposes and experiments not specifically disclosed herein.

One way to use the robotic workcell 1 under automated control of the main process, another is for substantial automated plasmid preparation. By example only, the illustrated method produces the end result of 20 DNA master tube racks with DNA suspended in water, 20 storage tube rack replicates of each culture plate, 20 isocard replicates of each culture plate and 1 multiplex tube rack with 96 colony samples in each of 96 tubes. By illustration in FIG. 1 of the preferred embodiment of the workcell 1, one way to reach this end result by way of example only is to first load two plate stacker elements 17 such as Hudson Control Group's StackLinks 17 with twenty 96-well deep-well plates labeled with bar codes and also load a colony picker element 9 with two plates containing bacterial colonies, scanning their bar codes. The robotic workcell 1 moves the 96-well deep-well plates from the plate stacker element 17 to either a pipetting robot element 15 or alternatively a liquid dispenser element 11, where 1.8 ml of growth medium broth is dispensed into each culture plate. The bar code on each culture plate is scanned by the bar code reader element 27 and then the plate is moved to the colony picker element 9 either by one or more track elements 7, 13, robotic arm element 3 or both depending upon the configuration of the workcell.

The colony picker element 9 picks one colony from the bacterial plates and loads one colony into each well of the culture plates. The machine does not pick the same colony twice. Therefore, after completion each well of the culture plate will have been inoculated with a different colony from the bacterial plate. The culture plate is then moved to a plate sealer element 21 via the robotic arm 3, track elements 7, 13 or both where the plate is sealed. The sealed plate then gets moved to an incubator/shaker such as the Liconic STX40 by the track elements 7, 13, robotic arm 3 or both. The plate is shaken and incubated for 18 to 24 hours.

After incubation the plates are moved to an advanced liquid handling system 15 such as Sias Xantus, where several functions are performed. First, 500 ul of glycerol are pipetted into each well of the 96 tube Matrix storage tube rack (60 stored on the deck of Xantus). Second, 3 ul are pipetted from the culture plate well into an isocard plate well (stored on the deck of Xantus). The isocard contains a barcode that is scanned and then the isocard is returned to the deck of Xantus. At the completion of the experimental run these isocards are unloaded from the deck of Xantus. Next, 250 ul of culture is pipetted into each tube of the Matrix rack for a unique 1:1 replicate of the culture plate. Finally, 5 ul from each well of culture plate is pipetted into a corresponding tube of a Matrix multiplex rack. Therefore, each tube will have one entire culture plate's 96 samples, and 20 tubes will hold the entire run's samples.

The Matrix storage rack is then moved to the plate sealer element 21 via the track element 7, 13, robotic arm 3 or both. From the plate sealer element 21 the storage rack is moved to a shaker, which may be an independent (not shown) or integrated shaker device, by way of track elements 7, 13, robotic arm 3 or a combination of both. At the shaker the plates are given a brief shake. After the brief shake, plates are moved to a plate stacker element 17 for manual removal from the system and freezer storage. Along the way, plates are either transported via the track elements 7, 13, robotic arm 3 or a combination of both and are again scanned by the barcode reader element 27 for purposes of tracking and organizing data.

From the Xantus liquid handling machine, the other culture plates from the 1:1 culture replication are moved into a centrifuge 31 such as the Ixion centrifuge. Culture plates are moved by track elements 7, 13, robotic arm 3, or a plate manipulator incorporated into the liquid handling machine or a combination thereof. The centrifuge 31 waits until two plates are loaded for balance purposes before beginning operation. When the culture plates arrive at the centrifuge 31 plate spinning begins. Plates are spun until the contents pelletize.

After spinning the culture plates, the plates are moved via the track elements 7, 13 robotic arm 3 or a combination of both to a machine which removes the media. Typically this is performed by a device with a disposal and waste system. An example of this type of machine is Hudson Control Group's ASP-96 Aspirator.

The next few steps require the plates to be moved between the 96-well aspirator 18 and the pipetting robot 15. Plates are moved between the two via a track element 7 or alternatively a robotic arm 3. Plates are first moved to the advanced liquid handling system such as the Sias Xantus. The liquid handling machine performs a series of functions. Plasmid preparation kit plates (such as Qiagen) are placed onto a shaker nest. 250 ul of P1 solution is added. The plates are shaken in order to resuspend the pellet contained at the bottom of the plate. Second, 250 ul of P2 solution is added to lyse the cells. The plates are shaken for an additional 4-5 minutes. Third, 350 ul of N3 solution is added and the plates are shaken for 15-20 seconds. Next, the plates are aspirated with the 96 well aspirator and then the plates are added to stacked filter plates on a vacuum nest. These plates are stored on the deck of the Xantus and moved to the vacuum nest as needed by the track element 7, robotic arm 3 or a combination of both. The vacuum is pulled for 1-2 minutes and the upper plate is discarded by delivery via a track element 7, robotic arm 3 or a combination of both to a plate stacker element 17, where the plates may be manually removed. The lower plate then moves to the upper nest, and PB wash solution is added by the pipetting robot 15. The wash solution is vacuum aspirated to waste for two minutes using the 96-well aspirator 18. PE wash solution is added by the pipetting robot 15. Then, the plate is vacuum aspirated for 1 minute by the aspirator 18. DNA is trapped in the filter frit.

Ethanol is dried from the tips of the filter plate. The empty Matrix DNA master tube rack is moved into the lower vacuum nest and the filter plate into the upper nest. This movement is performed by the track elements 7, 13, the robotic arm 3, a plate manipulator of the Xantus or a combination thereof. 75 ul of water are added to each plate so the DNA may dissolve. The solution is drawn down and 75 ul of water is added. The solution is drawn down immediately, causing the plasmid to transfer into the DNA master tube rack. The filter plate is then discarded. The water causes the DNA to dissolve or resuspend in the water solution within the master tube rack.

Barcodes on the DNA master tube rack are scanned by the barcode reader element 27 as the master tube rack is moved to the plate sealer element 21 via the track elements 7, 13, robotic arm 3 or both. The master tube rack is sealed by the plate sealer element 21. Plates are then moved to a plate stacker element 17 where they are stored and await manual removal from the system. This completes the run of the plasmid preparation to obtain the products in the amount specified above.

After completion of the plasmid preparation, the robotic workcell 1 may perform at least four other processes such as restriction mapping preparation, in vitro protein expression, sample assay and mass transformation of yeast. For example, one way a restriction mapping preparation may be performed is as follows.

At the completion of the plasmid preparation process described above the DNA master tube rack is moved to an advanced liquid handling system 15 such as the Xantus. Empty microplates are also moved to the Xantus. Depending upon the configuration of the machine, the capacity may vary. In the preferred embodiment 20 plates may be used in each run, although the system may be modified to allow for increased capacity. Plates are moved to the advanced liquid handler 15 via the track elements 7, 13, robotic arm 3 or a combination of both.

The Xantus advanced liquid handling system 15 will pipette 5 ul from each tube into each well of a microplate for a 1:1 replication. Next, 45 ul of reaction mix with enzyme is added to each well of the microplate to remove inserts from plasmids. Plates are then sent to the plate sealer element 21 via the track elements 7, 13, robotic arm 3 or both. There the plates are sealed. The plates are then sent to an incubator 19 such as the StoreX 100 incubator via the track elements 7, 13, robotic arm 3 or both. Plates will spend 1-2 hours in the incubator 19. Upon completion of the incubation, plates are moved to the plate stacker element 17 via the track elements 7, 13, robotic arm 3 or both. Plates may be stored at the plate stacker elements 17 and wait for manual removal from the system. A scientist may then remove the plates from the system and perform gel electrophoresis on the samples.

By way of example only, one way to perform in vitro protein expression on the robotic workcell 1 is as follows. At the completion of the plasmid preparation process described above the DNA master tube racks are moved to an advanced liquid handling system 15 such as the Xantus. This may be accomplished by the track elements 7, 13, the robotic arm 3 or both. Empty Matrix dilution tube racks are also moved to the same advanced liquid handling machine 15 such as the Xantus. The liquid handling machine 15 will pipette 50 ul from each tube of the master rack into each tube of the dilution rack. Then, 200 ul of water are added to dilute. The contents of the tubes are then mixed with a needle to ensure equal distribution.

The liquid handling machine 15 then adds 10 uL of the diluted plasmid preparation to 40 ul of in vitro expression mix (commercially available) to an empty microplate stored on a deck of the liquid handling machine 15 such as the Xantus. The pipette machine part of the liquid handler 15 will then pipette 10 ul from the dilution tube rack into each well of the microplate containing the protein mix to create a protein plate. The protein plate is then moved to the plate sealer element 21 via the track system 7, 13, robotic arm 3 or both. On the way to the plate sealer element 21 the protein plates, which contain bar codes, are scanned by the bar code reader element 27. The plate sealer element 21 then seals the plates. Prevention of evaporation is critical. After sealing the plates, the track elements 7, 13, the robotic arm 3 or both transport them to a shaker such as the Liconic STX40 incubator/shaker 19. Plates are incubated there at 30° C. for 100 minutes to produce the desired proteins. When the last plate has incubated for the full 100 minutes the incubator lowers the temperature to 4° C. for storage. All racks, plates and tubes in the workcell 1 including the DNA master and dilution tube racks may be moved to the plate stacker elements 17 by the track elements 7, 13, robotic arm 3 or both so that they may be removed from the system manually.

By way of example only, one way to perform a sample assay using the workcell 1 is as follows. At the completion of the protein expression procedure described above the protein plates are moved from the incubator 19, such as the Liconic STX40 incubator/shaker, via the track elements 7, 13, robotic arm 3 or a combination of both to an advanced liquid handling system 15 such as the Xantus.

The advanced liquid handling system 15 pipettes 50 ul from each well of protein plate to empty wells in a lidded assay microplate. The liquid handling system 15 will then add 250 ul of assay mix (this will vary by specific assay) to each well of the assay plate. The robotic workcell 1 will then move each plate to an incubator 19 such as the Liconic STX40 incubator. This is accomplished via the track elements 7, 13, robotic arm 3 or a combination of both. During transport the lidded assay microplates are scanned by the bar code reader elements 27. The plates are incubated in the incubator 19 for 15 minutes at 50-70° C.

After incubation, plates are shaken at 4° C. and then moved to an advanced liquid handling machine 15, such as the Xantus, by means of the track elements 7, 13, robotic arm 3 or a combination of both. Assay plates get 100 ul of stop solution added into each well by the advanced liquid handling machine 15. Assay microplates are then moved via the track elements 7, 13, robotic arm 3 or a combination of both to a plate reader element 25 such as the Bio-Tek® PowerWave HT reader. The data obtained from the plate reader element 25 is then merged with the DNA master tube IDs by the main process control 36 so that data may be understood and it can be determined which samples correspond to a particular well of the microplate being read by the plate reader element 25. Therefore, through recording results and the bar code tracking system a user may be able to determine which master DNA tube a sample has come from. The assay microplates may then be discarded from the system. This is done by the workcell 1 moving the microplates from the reader element 25 to a plate stacker element 17 via track elements 7, 13, robotic arm 3 or a combination of both. At the plate stacker element 17 the microplates may be removed from the system manually.

The protein plates are then moved from the shaker/incubator via the track elements 7, 13, robotic arm 3 or a combination of both to the advanced liquid handler 15 such as the Xantus. Clean assay plates are also moved from a plate stacker element 17 to the advanced liquid handler 15 by the same method. The advanced liquid handler 15 pipettes 5 ul from each well of the protein plate to a new assay plate. The advanced liquid handler 15 then pipettes, into the new assay plates, 25 ul of BioRad Solution A, waits 5 minutes and then adds 200 ul of BioRad Solution B. The new assay plates are then moved to the reader 25 which may be the Bio-Tek® PowerWave HT reader. This is done via track elements 7, 13, the robotic arm 3 or a combination of both and must be read by the plate reader element 25 within 15 minutes of adding BioRad Solution B. Plates are read and the data is merged with the DNA master tube IDS. The merging process is done by the computer 36 and data stored into the computer memory 38. The assay plates may be discarded after the reader element 25 has completed. Assay plates are moved to the plate stacker element 17 via the track element 7, 13, robotic arm 3 or a combination of both where they can be removed manually. Additionally, the protein plates may be removed from the system manually by moving them to the plate stacker element 17 via the track elements 7, 13, robotic arm 3 or a combination of both.

The robotic workcell 1 may also be used to perform a mass transformation of yeast. By way of example only, one way to perform this is as follows. After performing the plasmid preparation as described above, DNA master tube racks are moved to an advanced liquid handling machine 15 such as the Xantus. On the deck of the liquid handling machine 15 are clean PCR plates. The advanced liquid handling machine adds 125 ul of “competent yeast” mixture to each well of the clean PCR plates. The advanced liquid handler then pipettes 5-10 ul from each well of the DNA master tube rack into the PCR plate wells.

The workcell 1 then moves the newly filled PCR plate with transformation reactions to the thermal cycler machine 29. This is done via the track elements 7, 13, robotic arm 3 or a combination of both. Along the way the PCR plate wells are scanned using the bar code reader element 27. At the thermal cycler the samples are kept at 42° C. for 30-40 seconds. This “shocks” the yeast cells and enables the plasmids to enter the yeast cells.

The PCR plates are then moved from the thermal cycler machine 29 to a liquid dispenser element 11 via the track elements 7, 13, robotic arm 3 or a combination of both. One such liquid dispenser element 11 might be the Micro10 of Hudson Control Group and the liquid dispenser machine 29 adds 50 ul of clean YPD media to the PCR plates. The plates are then moved to an incubator/shaker 19 such as the Liconic STX40 where plates are shaken at 30° C. for 60 minutes to allow the yeast cells to recover from the heat shock treatment.

While PCR plates are being incubated for the 60 minutes of recovery time needed for the yeast, clean assay microplates are moved to the liquid dispenser element 11 via the track elements 7, 13, robotic arm 3 or a combination of both. Xylose media is added to each well of the microplates. At the completion of the 60 minute incubation period, the PCR plates and the microplates filled with the new yeast-selective medium are both moved to a colony picker workcell 9. A colony picker workcell 9 that may be used for this process is the BioRad Versarray. Plates are moved to the colony picker 9 via the track elements 7, 13, robotic arm 3 or a combination of both.

The colony picker workcell 9 will then inoculate the microplates, filled with the new yeast-selective medium, with cells from the PCR plate. On the Versarray colony picker 9 this is accomplished through the use of the 96-pin head. The PCR plate may then be either placed into an incubator calibrated to freezing temperature for storage or may be transported to plate stacker elements 17 for manual removal. Transport is done via the track elements 7, 13, robotic arm 3 or a combination of both. The newly inoculated plate is moved to a shaking incubator such as the Liconic STX40 for 5 hours at 30° C. After the incubation period, the newly inoculated plate is moved to a plate reader element 25 such as the Bio-Tek® PowerWave HT. The plate is moved via the track elements 7, 13, robotic arm 3 or a combination of both. The plate reader element 25 scans the samples for turbidity and data is merged with the DNA master tube IDs from the previous plasmid preparation. The data may also be stored on a hard drive or printed so that results may be read and interpreted or experimental runs may be tracked.

The process above describing protein expression may be for the purposes of identification of novel codon patterns from a cDNA library in an effort to optimize the activity of certain genes in targeted cells. This process involves the activities described above, wherein the initial DNA inserts consist of wild-type and/or mutant clones including analogues of wild-type genes, from cDNA libraries or other sources of organisms that exhibit the desired expression properties. Analog means DNA molecules that are structurally similar to another but differs slightly in composition, usually a single element. Therefore, an analogue may have a single deletion, insertion, substitution or modification. A mutant on the other hand, may have more than one deletion, insertion, substitution or modification.

A wild-type open reading frame (ORF) from a cDNA library may be cloned into a plasmid vector for expression in prokaryotic or eukaryotic cells. Alternatively, the wild-type ORF may be modified or mutagenized to create an analogue or mutant and then cloned into a plasmid vector for expression in yeast, bacteria, fungus, plant, or animal cells for expression of the respective protein. The wild-type ORFs and respective analogues and/or mutants may then be analyzed and compared to one another for protein expression using the robotic workcell 1.

The method of the present embodiment also deals with the concept of assembling a wild-type ORF or introducing one or more modification(s) into an ORF that will result in some functional modification of the expressed protein and may be implemented with the assistance of the automated workcell system as described herein. The invention generally entails cloning of a wild-type ORF, mutant or an analogue thereof or a nucleic acid, e.g., DNA or RNA encoding for the protein of interest. Analogues and mutants may also occur at the nucleic acid level and amino acid level. Therefore, changes such as additions, insertions, deletions or modifications may occur in DNA, RNA and amino acid polypeptide chains.

In preferred embodiment the analogs and mutants differ by one or more nucleic acid addition, deletion, insertion or substitution from that of the wild-type nucleic acid sequence. Such changes in the ORF sequence, whether resulting from random, site-directed and/or evolutionary mutagenesis, that give rise to alterations in the amino acid sequence can 1) give no change from that of the wild-type in the activity of the mutant protein being expressed, 2) remove all or part of the wild-type activity in the protein being expressed, 3) enhance the activity of the mutant over that of the wild-type, or 4) more commonly yield mutant expressed proteins with activities falling between case 2 and case 3, depending on the number of codons involved. It is well understood by the skilled artisan that there is a limit to the number of changes that may be made within a portion of the nucleic acid sequence and/or amino acid sequence and still result in a molecule or protein with an acceptable level of equivalent biological activity or function. It is also well understood that where certain nucleic acids or amino acids are shown to be particularly important to the biological or structural properties of a polypeptide, such residues may not generally be changed. For example, the hydrophobicity, hydrophilicity, charge, size and the like of the corresponding amino acid to the nucleic acid sequence all influence the success of nucleic acid and/or amino acid changes. The nonpolar (hydrophobic) amino acids include alanine, leucine, isoleucine, valine, proline, phenylalanine, tryptophan and methionine. Amino acids containing aromatic ring structures are phenylalanine, tryptophan, and tyrosine. The polar neutral amino acids include glycine, serine, threonine, cystein, tyrosine, asparagine, and glutamine. The positively charged (basic) amino acids include arginine, lysine and histidine. The negatively charged (acidic) amino acids include aspartic acid and glutamic acid.

Therefore, based upon these considerations, arginine, lysine and histidine; alanine, glycine and serine; and phenylalanine, tryptophan and tyrosine; are defined herein as biologically functional equivalents. To effect more quantitative changes, the hydropathic index of amino acids may be considered. Each amino acid has been assigned a hydropathic index on the basis of its hydrophobicity and charge characteristics, which are as follows: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). The importance of the hydropathic amino acid index in conferring interactive biological function on a protein, and correspondingly a polyamino acid, is generally understood in the art. It is known that certain amino acids may be substituted for other amino acids having a similar hydropathic index or score and still retain a similar biological activity. In making changes based upon the hydropathic index, the substitution of amino acids whose hydropathic indices are within 2 is preferred, those which are within approximately 1 are particularly preferred, and those within approximately 0.5 are even more particularly preferred.

It is also understood in the art that the substitution of nucleic acids and corresponding like amino acids can be made effectively on the basis of hydrophilicity. As disclosed in U.S. Pat. No. 4,554,101, the following hydrophilicity values have been assigned to amino acid residues: arginine (+3.0); lysine (+3.0); aspartate (+3.0±1); glutamate (+3.0±I); serine 5 (+0.3); asparagine (+0.2); glutamine (+0.2); glycine (0); threonine (−0.4); proline (−0.5 ±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). In making changes based upon similar hydrophilicity values, the substitution of amino acids whose hydrophilicity values are within ±2 is preferred, those which are within ±1 are particularly preferred, and those within ±0.5 are even more particularly preferred.

The wild-type, mutants and/or analogues of nucleic acid and/or amino acids may be produced recombinantly or synthetically, preferably recombinantly, by standard techniques. Aside from production and isolation of the nucleic acids from cDNA libraries, nucleic acids and polypeptides may be recombinantly produced in bacterial, mammalian, and yeast cells, e.g., E. coli, Streptomyces, Bacillus subtilis, fungal cells such as yeast and animal cells such as mammalian cells. Choice of appropriate vectors, promoters and other 5′ and 3′ regulatory flanking sequences, e.g., origin of replication, translation initiation and termination, leader sequence, marker genes, methods of introducing the DNAs encoding for polypeptides into the host cell, culturing, isolation and purification techniques, are all well known in the art. Cell-free translation systems may also be employed.

Nucleic acids encoding the wild-type, mutants and analogs thereof of the present invention may be prepared in accordance with standard procedures such as cloning or synthetic synthesis. In addition to the nucleotide sequence wild-type proteins, the portion of the full-length polynucleotide that encodes the analogue may be easily designed by introducing a “stop” codon at the 3′ end. By this process, adding stop codons at different places at the 3′ end of a wild-type ORF, allows for incrementally synthesizing progressively larger segments, mutants and analogues of the wild-type ORF.

Each wild-type, mutant and analogue gene attained from a cDNA library may then be expressed. These segments may be expressed in an expression system. The expression system used will depend upon experimentation being performed and the type of gene being studied. For example, a particular gene may be expressed in vivo or in vi tro and a gene may be expressed in different types of cells such as yeast, fungal or other types of prokaryotic or eukaryotic cells. Expression systems are well known to a person skilled in the art.

Expression of the desired protein product is evaluated by measuring the biochemical activity and/or binding site recognition of each of the segments of either wild-type or analogues thereof. Measurements may be done with standard laboratory equipment such as a plate reader element 25 or mass spectrometer. Binding site recognition may be evaluated by performing assays tailored to the specific enzyme or protein product encoded for by the gene or alternatively the specific selectable marker product encoded for within the cell. Further evaluations may be determined using what is known in the art with respect to the properties of the amino acids discussed above.

Based on the measured biochemical activity it may be determined that a specific wild-type, mutant or analogue thereof shows exceptional activity of the desired function. This process may consist of a selection so that only the fragments with desired functions are seen. After selection, the fragment may be isolated and further genetic studies may be performed to ensure accuracy. Fragments may then be introduced into a yeast, fungal, other eukaryotic cell or prokaryotic cell for determination of optimal gene activity. Alternatively, the ORF fragment may be expressed in vitro with techniques known by those skilled in the art.

The ORF segment, which may be a wild-type, mutant or an analog segment, may be created by incrementally synthesizing the wild-type gene derived from a cDNA library. Alternatively, these ORF segments may be synthesized by means of amplifying overlapping oligomers. The oligomers collectively may represent the sequence of the entire ORF. Analogues including truncated forms of an ORF may also be synthesized in a similar manner. Moreover, ORF segments may be synthesized in any direction based on the ORF wild-type sequence or where at least one segment has an introduced modification such as an addition, insertion, or deletion or substitution.

The present embodiment may be used to produce a clone that has one or more desirable functional modifications of an ORF. This is accomplished by first incrementally synthesizing progressively larger segments of an ORF in any direction on the ORF, either on the wild-type sequence or where at least one segment contains a first introduced modification. The introduced modification may be an addition, deletion, insertion or substitution. The segment is then expressed in an expression system which may be in vitro or in vivo and may include systems such as yeast, fungal, other eukaryotic cells or prokaryotic cells. The biochemical activity and/or binding site recognition of each segment is measured. A selection for the desirable functional modification is made based on the biochemical activity and/or binding site recognition of the segment with this first introduced modification in the ORF. The segment selected may be either wild-type or alternatively may be a segment with at least one introduced modification including deletions, insertions, addition and substitutions.

Progressively, larger segments of the ORFs are incrementally synthesized. With these larger segments a second modification is introduced. The second introduced modification may be an addition, deletion, insertion or substitution. Again, the newly synthesized ORF is expressed in an expression system which may be in vitro or in vivo and may include systems such as yeast, fungal, other eukaryotic cells or prokaryotic cells. The biochemical activity and/or binding site recognition of each segment are measured. A selection for the desirable functional modification is made based on the biochemical activity and/or binding site recognition of the segment with the first and second introduced modifications in the ORF.

The method for producing a clone described above may be continued in a repetitive manner to produce a clone having a plurality of desirable functional modifications of an ORF. The process discussed here may be repeated however many times necessary in order to produce the desired number of modifications resulting in the desired segment of the ORF. For example, after producing a clone with at least one desirable functional modification from a wild-type, mutant or modified ORF an additional modification may be selected for by performing random mutagenesis, directed mutagenesis, and evolutionary mutagenesis. Applicants do not wish to be bound by any particular mechanism or process by which the mentioned types of mutagenesis are performed.

The method herein described generally includes screening for desired biochemical activity and/or binding site recognition for high solubility and greater ability to be purified from aqueous fraction. The desirable biochemical activity and/or binding site recognition may also generally be screened by detecting a reduction in formation of cysteine bridges by systematic removal of all cysteine amino acids in the ORF.

The ORF segments may encode a reduced number of translational stops when expressed in a heterologous expression system as compared to a wild-type ORF of the same gene. Such a segment may additionally be a truncated form of a wild-type ORF of the gene. The segment may also be a mixture of wild-type and modified segments of sequence in the assembled ORF. Through the method herein described the entire ORF becomes optimized by incorporating the codons most frequently used within a particular organisms expression system.

The before mentioned methods, i.e. the concept of introducing multiple modifications into an ORF, may be accomplished without being limited to incrementally synthesizing progressively larger segments of the ORF. That is the steps of the process may also begin with a cDNA library. Therefore, this method may be used for obtaining a representative ORF for each gene (all or partial) in the genome of an organism (prokaryotic or eukaryotic), wherein the genes are derived from collections of cDNAs in various previously generated libraries.

For example, an ORF for each gene in the cDNA library capable of being expressed in an expression system may be provided in bacterial colonies. The ORFs from the cDNA library are expressed in an expression system. The system may be an in vitro or in vivo system such as yeast, fungal, other eukaryotic cells or prokaryotic cells. The cDNA libraries may then be screened for the expressed proteins and the desired function in vitro or in vivo by determining binding and/or biochemical activity. Then, biochemical activity and/or binding site recognition of the ORFs from the cDNA library may be determined. Modifications in at least one ORF from the cDNA library may be selected wherein the modification resulted from either random mutagenesis, directed mutagenesis or evolutionary mutagenesis. Subsequently, the desired functional modifications may be selected based on the biochemical activity and/or binding site recognition of the expressed ORFs.

The present invention may be used for transforming an entire library of cDNAs having a representative ORF for each gene (all or partial) in the genome being expressed in yeast, fungal, other eukaryotic cells or prokaryotic cells. This means that sets of assembled or wild-type or modified ORFs or cDNA library ORFs may ultimately be transformed into a cellular system for strain or cell line improvement. An ORF is provided for each gene in a cDNA library capable of being expressed in prokaryotic and/or eukaryotic cells. The ORF is expressed in an expression system including in vivo or in vitro systems. The cDNA libraries are screened for desired function in vivo or in vitro by determination of binding and/or biochemical activity. The biochemical activity and/or binding site recognition of the cDNA library ORF is determined. The cDNA libraries for high throughput transformation of bacterial, fungal and yeast strains, and/or transfection of plant and mammalian cell lines are either wild-type, random mutagenized, targeted mutations, and or evolutionary mutations present in combinations or separately.

High throughput may be used to asses the ORFs and transformed bacterial, fungal and yeast strains, and/or transfected plant and mammalian cell lines. This may be done using functional growth assays or biochemical assays for identification of optimal strains and expressed protein improvements. Henceforth, ORFs with optimal characteristics may be selected for with this method.

The present invention of the automated plasmid-based proteomics workcell 1 may be used for modifying an ORF of a gene so that the expression product of the ORF is characterized by a desirable functional modification. The automated laboratory system, such as the workcell 1 described previously, incrementally synthesizes a plurality of progressively larger segments of an ORF. At least one of the ORF segments includes a modification. Such modification may be an addition, insertion, deletion, substitution or truncation. The segments are simultaneously expressed in an expression system and then the biochemical activity and/or binding site recognition of each of the segments is determined. At least one of the segments characterized by the desirable functional modification based on the biochemical activity and/or binding site recognition is selected. The plurality of progressively larger segments of the ORF is at least more than one combination. Expression of ORFs is either from cDNA libraries or from modified ORFs. The production of new prokaryotic strains and/or eukaryotic cell lines is performed by mass introduction of a cDNA library or combinations of libraries.

The automated system may be used to introduce modifications such as insertions, deletions, additions, substitutions or truncations into an ORF and select for a resulting clone that expresses a desirable functional trait. The automated system may modify an ORF of a gene so that the expression product of the ORF is characterized by a desirable functional modification or combination of function modifications.

A plurality of clones is provided that have an introduced modification in the ORF as compared to a wild-type form of the ORF. The plurality of clones is mutagenized in order to introduce at least one additional modification into the ORF of each one of the plurality of clones. This might be accomplished by any known technique in the art such as chemicals, UV light, evolutionary, site-directed, or random mutagenesis. The additional modifications in the ORFs are simultaneously expressed in each one of the clones in an expression system. The expression system may be in vitro or in vivo such as yeast, fungal, other eukaryotic or prokaryotic system. The biochemical activity and/or binding site recognition of each of the clones are determined. Clones having the desirable functional modification or combination of functional modifications based on the biochemical activity and/or binding site recognition of the clone are selected.

Once the clones producing the best expression results have been selected, and their novel codon patterns identified, the workcell system 1 would re-introduce just those clones into the system by assembling them from pre-prepared oligonucleotides that include the codon patterns of the mutated open reading frames identified above. These oligonucleotides would be delivered to the system's thermal cycler element 29 by either the track elements 7, 13, robotic arm 3 or both to perform PCR reactions. The PCR reaction, when run in this circumstance, will form double stranded DNA templates. The double stranded DNA templates may then be introduced or inserted into plasmids.

The robotic workcell system 1 may be used to perform a mass transformation or transfection of the plasmids containing the clones obtained by the aforementioned methods into prokaryotic or eukaryotic cells for in vivo expression of desired proteins. Alternatively, expression of those proteins may be performed in vitro, as may be required by particular clone types which cannot be expressed in vivo. Expression is done in order to identify the mutations of the wild-type gene that may give rise to optimal expression levels and activity in the targeted cell. For example, clones may be transformed into E. coli, grown on growth dishes and processed through the colony-picking, incubation and plasmid preparation processes described above. Sequential PCR may then be performed so that the clones may be placed back into the thermal cycler element 29 for the addition of the next open reading frame onto one of the cDNA strands via PCR reaction.

The process of sequential PCR to form ever longer assembled DNA strands followed by plasmid preparation, transformation, colony growth, picking and plasmid preparation would continue until, as one possible outcome of the invention, a full-length gene has been assembled, including the mutant codon patterns at the appropriate sequential location. It can also be used for assembly of wild type sequences from bioinformatic database collections or to build any gene of a known or desired codon sequence pattern. This technique would be useful to construct, for instance, a gene to produce a therapeutic agent of known codon sequence that is not found naturally in the targeted cell using the targeted cell's genetic mechanisms.

When full-length genes have thus been assembled, these would then be transformed/transfected into the targeted cell line, either singly or in combination, using the integrated instruments of the above described workcell 1 system, particularly the automated delivery of plates among its colony picker 9, incubator 19, robotic pipettor 15 and thermal cycling instrument 29, utilizing the system's capabilities to perform the several stages of the assembly and transformation/transfection processes simultaneously. For example, the instruments described are all used in the processes of plasmid preparation, restriction mapping preparation, in vitro protein expression, in vivo protein expression, sample assay and mass transformation of cells. All of these processes are described in detail above. The overall objective of this process would be to produce a cell with the desired improved protein expression characteristics and cellular traits or more specifically a gene or polypeptide encoding for the improved expression characteristics for that particular gene, giving rise to new high-value proteins, protein arrays, antibody arrays, tractable drug targets, biologicals, cell-derived bioproducts and biocatalysts that can produce new chemical entities.

Although the invention herein has been described with reference to particular embodiments, it is to be understood that these embodiments are merely illustrative of the principles and applications of the present invention. For example, the robotic workcell 1 is modular and expandable, allowing for a variety of configurations to be designed to meet any lab's specific automation needs. These can range from simple workcells that automate the process to full-scale systems that can automate entire proteomics assays. Moreover, while the above described system has been illustrated as having centralized scheduler which periodically or continuously polls the instruments of the workcell for tracking status of operations on microplates and scheduling further procedures permitting pipelining of such procedures with the instruments, such a system may be implemented by a more simple non-centralized approach by which each instrument/robot/conveyor of the sequential process workcell automatically responds to the presence of a sample plate entering its workspace or input area, performing its designed task and moving the sample to its output location that corresponds with an input location of the next instrument/robot/conveyor of the workcell. It is therefore to be understood that numerous modifications may be made to the illustrative embodiments and that other arrangements may be devised without departing from the spirit and scope of the present invention as defined by the appended claims.

1. A robotic workcell system for open reading frame assembly and expression in a sample holder and for protein evaluation comprising:
a plurality of instruments including an automated colony picking device, each instrument configured to perform an automated process with a sample of a sample holder; and
one or more automated sample holder transport units configured for conveying a sample holder to and from each of the instruments of the plurality of instruments,
wherein the plurality of instruments further includes an automated liquid handler device or automated pipetting device.

2. The robotic workcell system of claim 1 further comprising a processor configured with control instructions to control sample holder processing by the plurality of instruments and to control sample holder movement between the plurality of instruments by the one or more automated sample holder transport units.

3. The robotic workcell system of claim 1 wherein the plurality of instruments further includes an automated PCR thermocycler device.

4. The robotic workcell system of claim 2 further comprising a reader element configured for access by the one or more automated sample holder transport units and to read data from a sample holder moved under control of the processor.

5. The robotic workcell system of claim 2 wherein the one or more automated sample holder transport units comprises a multi-axis robot sample holder handler configured to transfer sample holders under control of the processor.

6. The robotic workcell system of claim 2 wherein the one or more automated sample holder transport units comprises a conveyor track to transfer sample holders under control of the processor.

7. The robotic workcell system of claim 1, wherein the plurality of instruments includes an incubator device.

8. The robotic workcell system of claim 4, wherein the plurality of instruments includes a microplate washer, seal piercer, and filler device.

9. The robotic workcell system of claim 7, wherein the plurality of instruments includes a plate sealer device.

10. The robotic workcell system of claim 6 wherein the one or more automated sample holder transport units further comprises a multi-axis robot sample holder handler configured to transfer sample holders to or from the conveyor track.

11. The robotic workcell system of claim 1 wherein the processes of the plurality of instruments comprises an automatic method of bacterial or yeast colony processing from initial plated colony growth through plasmid assay including the steps of:
picking cells into microplate wells;
sample data archiving;
plasmid preparation;
plasmid quality analysis;
plasmid reaction; and
plasmid assay.

12. The robotic workcell system of claim 2 wherein the processor is configured with control instructions for controlling the plurality of instruments to control an automatic method comprising the steps of:
picking cells into microplate wells;
sample data archiving;
plasmid preparation;
plasmid quality analysis;
plasmid reaction; and
plasmid assay.

13. The robotic workcell of claim 2, further comprising a bar code reader device, a bar code tracking element, and a database for storing data collected by said reader.

14. The robotic workcell system of claim 1, wherein the plurality of instruments includes an incubator device and wherein the automated colony picker device and the liquid handler device are on a first track of the one or more automated sample holder transport units, and the colony picker element and the incubator device are on a second track element of the one or more automated sample holder transport units.

15. The robotic workcell system of claim 1, wherein one or more of the automated processes of the plurality of instruments comprises an automated preparation of plasmids for use in genomic or proteomic processes.

16. The automated workcell system of claim 2 wherein the control instructions further comprise a scheduler configured to schedule simultaneous processing of microplates by a plurality of instruments of the workcell.

17. An automated workcell system for open reading frame assembly and expression in a microplate and for protein evaluation comprising:
an automated colony picking device;
an automated temperature controlled incubator;
one or more automated microplate transfer units configured to convey microplates to and from the colony picking device and the incubator.

18. The automated workcell system of claim 17 further comprising a controller configured to control microplate processing by the automated colony picking device and the automated incubator and further configured to control microplate movement between the colony picking device and the incubator by controlling the one or more automated microplate transfer units.

19. The automated workcell system of claim 18 further comprising an automated liquid-handling device wherein the controller is further configured to control microplate processing by the automated liquid-handling device and is further configured to control microplate movement to and from the automated liquid-handling device by the one or more automated microplate transfer units.

20. The automated workcell system of claim 18 further comprising an automated PCR thermocycler device wherein the controller is further configured to control microplate processing by the automated PCR thermocycler device and is further configured to control microplate movement to and from the automated liquid-handling device by the one or more microplate conveyor units.

21. The automated workcell system of claim 18, further comprising a multi-axis robot microplate handler, wherein the controller controls movement of the microplate by the multi-axis robot microplate handler.

22. The automated workcell system of claim 19 further comprising a microplate reader device where then controller is configured to track processing of microplates by the system in association with data generated with the microplate. label reader device.

23. The automated workcell system of claim 22 wherein the controller comprises a scheduler configured to schedule simultaneous processing of microplates by a plurality of instruments of the workcell.

24. A method of a controller for an automated laboratory workcell system for processing bacterial or yeast colonies by instruments of the workcell comprising steps of:
automatically picking colony growth to microplate wells;
automatically performing a plasmid preparation;
automatically performing a plasmid quality analysis;
automatically performing a plasmid-based reaction; and
automatically performing a plasmid-based assay,
wherein said steps are performed in conjunction with controlling automatic transporting of at least one sample holder between the instruments of the laboratory workcell such that said steps of the workcell are automatically performed on at least one sample of the at least one sample holder.

25. The method of claim 24 further comprising automatically recording data associated with the at least one sample, said data associated with each of said steps performed by the automated laboratory workcell system.
